(19) United States
(12) Patent Application Publication
(10) Pub. No.: US 2002/0098201 A1
(43) Pub. Date: Jul. 25, 2002

(54) Novel myxoma genes for immune modulation

(76) Inventor: Grant McFadden, London (CA)

Correspondence Address: CLARK & ELBING LLP, 176 FEDERAL STREET, BOSTON, MA, 02110-2214, US

(21) Appl. No.: 10/010,901

(22) Filed: Dec. 6, 2001

Related U.S. Application Data
(62) Division of application No. 09/615,041, filed on Jul. 12, 2000.
(60) Provisional application No. 60/143,317, filed on Jul. 12, 1999.

(51) Int. Cl. C12Q 1/70; C07H 21/04; C12P 19/34; A61K 39/12; C12N 5/06; C12N 5/16

(52) U.S. Cl. 424/204.1; 435/5; 435/343.2; 435/91.33; 536/23.72

(57) ABSTRACT

The present invention provides novel genes of the viral genus leporipox that encode novel immunomodulatory proteins. The present invention also provides therapeutic compositions containing these novel polypeptides and methods for use for treating a variety of inflammatory and autoimmune disorders. In one preferred embodiment, the present invention provides methods for producing the novel immunomodulatory polypeptides in a substantially pure form. In another preferred embodiment, the invention provides methods for treating individuals diagnosed with autoimmune or inflammatory disorders by gene therapy. In yet another preferred embodiment, the methods and compositions of the present invention may be used to treat cancer. Finally, the invention provides methods to identifying and isolating additional leporipox virus immunomodulatory polypeptides.

This application is a divisional of and claims priority from U.S. patent application Ser. No. 09/615,041, filed on Jul. 12, 2000, which claims priority from U.S. provisional application serial No. 60/143,317, filed on Jul. 12, 1999.

### FIELD OF THE INVENTION

This invention relates generally to the field of immunology and specifically to novel viral genes and proteins and methods for use of the same as immune modulators.

### BACKGROUND OF THE INVENTION

Viruses propagate by living within cells of higher-order vertebrates. Accordingly, they have evolved to specifically avoid the host immune system. In fact, virus survival is dependent upon strategies which can evade, suppress, counteract, or otherwise circumvent the myriad of host responses to a foreign invader. The selective pressure conferred by the effector elements of the immune system of the host can clearly be a powerful element of evolutionary pressure; in fact all eukaryotic viruses existing today contain imprints or remnants of their battles with the immune system as evidenced by the presence of encoded proteins that suppress the immune system or as evidenced by survival strategies that allow the virus to evade immune system detection.

The specific strategy or strategies used by a virus varies dramatically according to its genome capacity. Viruses with small genomes ensure their survival by exploiting weaknesses or gaps in the host immune repertoire to evade detection. Alternatively, the smaller genome replicates very rapidly, effectively outpacing the immune response. The larger DNA viruses (i.e. the adenoviruses, herpesviruses, iridoviruses and poxviruses) specifically encode proteins that function to protect the virus from immune recognition and/or clearance by the infected host. Such “subversive” viral proteins are useful therapeutics for the treatment of inflammatory and auto-immune disorders.

Recent studies on the myxoma virus, a virus of the genus leporipox, which also includes Shope Fibroma virus, have shown that these viruses are among those that disrupt the immune system by a variety of strategies. Myxoma virus is the infectious agent of a virulent systemic disease of domestic rabbits called myxomatosis. Originally described in the last century, myxoma was the first viral pathogen discovered for a laboratory animal and was the first viral agent ever deliberately introduced into the environment for the explicit purpose of pest eradication. Since its release into the Australian and European feral rabbit populations more than 40 years ago, the field strains of both the rabbit and virus have been subjected to mutual evolutionary and selective pressures that have resulted in a steady-state enzootic in the inoculated areas (Fenner, F. and Ratcliffe, F. N., “Myxomatosis,” Cambridge University Press, London, 1965).

Myxoma shares many of the biological features associated with poxviruses, namely cytoplasmic location of replication and a large double stranded 160 kilobase DNA genome. Multiple lines of evidence indicate that myxoma, like poxviruses, encodes multiple gene products whose function is to permit the spread and propagation of the virus in a variety of host tissues. Some of these viral proteins interact with known components of the host's immune system or interfere with intracellular signal transduction to specifically counteract or subvert the development of the host inflammatory response and acquired cellular immunity. Poxviruses in general have been a rich source of such immunomodulatory proteins.

Examples of such immunomodulatory gene products include myxoma growth factor (MGF), which stimulates neighboring cells in a paracrine-like fashion via the cellular epidermal growth factor receptor; Serp 1, a secreted glycoprotein with serine protease inhibitor activity that prevents development of the early inflammatory response; T1, a soluble scavenger of CC chemokines that plays a central role in the host defense against virus infection; T7, a secreted viral homologue of the cellular interferon-γ receptor, that binds and inhibits rabbit interferon-γ; and M11L, a surface receptor-like protein that interferes within the inflammatory response by an unknown mechanism. The SERP-1 protein has been used successfully as an anti-inflammatory agent.

Host and viral immunomodulatory proteins include chemotactic cytokines, called “chemokines.” Chemokines are small molecular weight immune ligands that are chemoattractants for leukocytes, such as neutrophils, basophils, monocytes, and T cells. There are two major classes of chemokines, both of which contain four conserved cysteine residues that form disulfide bonds in the tertiary structure of the proteins. The α class is designated C-X-C (where X is any amino acid), and includes 11-8, CTAP-III, gro/MGSA and ENA-78; and the β class, designated C-C, includes MCP-1, MIP-1α and β, and Regulated on Activation, Normal T Expressed and Secreted protein (RANTES). The designations of the classes are according to whether an intervening residue spaces the first two cysteines in the motif. In general, most C-X-C chemokines are chemoattractants for neutrophils, but not monocytes, whereas C-C chemokines appear to attract monocytes, but not neutrophils. Recently, a third group of chemokines, the “C” group, was designated by the discovery of a new protein called lymphotactin. The chemokine family is believed to be critically important in the infiltration of lymphocytes and monocytes into sites of inflammation.

Both the host and virus may also encode a class of proteins that regulate apoptosis (or cell death). The relationship between viruses and apoptosis is complex, in part because all viruses are faced with the need to first productively replicate within a receptive intracellular environment, then to spread to other cells or tissues, and finally to seek out new hosts in order to continue the chain of propagation. Thus, there are times when the premature induction of apoptosis can be detrimental to the virus, for example, by disrupting the viral replicative life cycle, while at other times apoptosis can become advantageous, for example, by facilitating virus dissemination and secondary infection of phagocytes or other immune cells. As viruses have acquired mechanisms to evade the host's immune system, viruses have collectively evolved strategies to either inhibit or stimulate apoptosis, depending on the particular virus/host interaction.

There exists a need for new drugs that are useful for the treatment of human inflammatory and autoimmune disorders. Identification of novel immunomodulatory agents may reveal new pharmaceuticals that can be used to suppress inflammation and dysregulation of the immune system. In addition, novel agents provide new probes and targets that can be used to identify novel elements of the cellular immune repertoire and new classes of drugs. Such tools may promote understanding of the molecular mechanisms by which viral immunomodulators function and may reveal targets for pharmacological intervention that could be of substantial benefit in treating these diseases.

### SUMMARY OF THE INVENTION

In general the present invention provides novel nucleic acids encoding novel immunomodulatory proteins that may be used as therapeutic immunomodulatory proteins for the treatment of a variety of immunomodulatory disorders.

In one aspect, the invention provides a substantially pure leporipox virus immunomodulatory polypeptide, that may be derived from, for example, myxoma virus or Shope fibroma virus. In preferred embodiments, the polypeptide can encode a chemokine, a cytokine (e.g. SEQ ID NO:32), an immunomodulator, an anti-inflammatory polypeptide, an immunoreceptor, or multi-transmembrane receptor protein, or have an identifiable signal sequence. In other preferred embodiments, the polypeptide can include an amino acid sequence selected from the group consisting of SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, and SEQ ID NO:40.

In another aspect, the invention provides a substantially pure leporipox virus nucleic acid molecule (for example, genomic DNA, cDNA, or synthetic DNA, or mRNA) comprising a sequence encoding a leporipox virus polypeptide, e.g., a myxoma virus polypeptide or a Shope fibroma virus polypeptide. In a preferred embodiment, the nucleic acid molecule encodes a polypeptide including an amino acid sequence that is substantially identical to an amino acid sequence selected from the group consisting of SEQ ID NO:21, SEQ ID NO:22, SEQ ID NO:23, SEQ ID NO:24, SEQ ID NO:25, SEQ ID NO:26 SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, and SEQ ID NO:40. In other preferred embodiments, the nucleic acid molecule can be genomic DNA, cDNA, and mRNA. In other preferred embodiments, the nucleic acid molecule can encode a polypeptide with an identifiable signal sequence, a chemokine, a cytokine (e.g., (SEQ ID NO: 32), an immunomodulator, an anti-inflammatory polypeptide, an immunoreceptor, or a multi-transmembrane receptor protein.

The invention also provides a vector (e.g., a gene therapy vector) including the leporipox virus nucleic acid molecule; a cell (e.g., a bacterial, yeast, nematode, or mammalian cell, such as a human cell or a rodent cell), that may include the vector; a non-human transgenic animal including the leporipox virus nucleic acid molecule; a non-human transgenic animal having a knockout mutation in one or both alleles encoding a polypeptide substantially identical to a leporipox virus polypeptide; and a cell from the non-human transgenic animals. In a preferred embodiment, the nucleic acid molecule of the vector is operably linked to regulatory sequences, including a promoter, for expression of a leporipox virus polypeptide. The promoter can be the promoter native to the leporipox gene. In addition, transcriptional and translational regulatory regions are preferably native to a leporipox gene. A constitutive promoter or an inducible promoter is also included in the invention. In related aspects, the invention features substantially pure nucleic acid encoding truncated and augmented leporipox polypeptides substantially identical to those described in FIGS. 21-40.

In related aspects, the invention features a transgenic animal containing a transgene which encodes a leporipox virus polypeptide and methods of using the leporipox virus nucleotide sequence to engineer a transgenic animal having a knockout mutation in the leporipox gene. For example, the invention provides transgenic animals having a loss of function mutation in a gene substantially identical to a leporipox virus polypeptide. In a preferred embodiment of the present invention, the loss of function mutation is in any of the genes of FIGS. 1-20 that encode the proteins of FIGS. 21-40. The invention also features a cell that expresses a leporipox virus gene (i.e., a transgenic cell). In various embodiments, the cell is a transformed animal cell.

In other related aspects, the cell that expresses a leporipox virus gene is present in a patient needing treatment in the form of immunomodulation (e.g., immunosupprssion or immunostimulation). Alternatively, the patient is experiencing any of a wide variety of inflammatory or autoimmune disorders (e.g., an allergic reaction, acute inflammation, rheumatoid arthritis transplant rejection, restenosis, asthma, uveitis or inflammatory bowel disease).

In another aspect, the invention provides a nucleic acid molecule having at least 50% nucleotide sequence identity to a sequence encoding a leporipox virus polypeptide or fragment thereof, where the fragment includes at least six amino acids, and the nucleic acid molecule hybridizes under high stringency conditions to at least a portion of a leporipox virus nucleic acid molecule. In a preferred embodiment, the nucleic acid molecule has 100% complementarity to a nucleotide sequence encoding a leporipox virus polypeptide or fragment thereof, where the fragment includes at least six amino acids, and the nucleic acid molecule hybridizes under high stringency conditions to at least a portion of a leporipox virus nucleic acid molecule.

In another aspect, the invention provides a nucleic acid molecule that includes a sequence that is antisense to the coding strand of a leporipox virus nucleic acid molecule or a fragment thereof.

In another aspect, the invention provides an antibody that specifically binds to a leporipox virus polypeptide. In a preferred embodiment of this aspect, the polypeptide comprises an amino acid sequence that is substantially identical to an amino acid sequence selected from the group consisting of SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, and SEQ ID NO:40. In another aspect, the invention features a purified antibody that binds specifically to a murine or human leporipox protein. Such an antibody may be used in any standard immunodetection method for the identification of a leporipox polypeptide. Such an antibody may also be used to inhibit leporipox protein function and to predict prognosis following tumor diagnosis. In various embodiments, the antibody may be an intact monoclonal or polyclonal antibody, but may also be an immunologically-active antibody fragment, such as an Fab′ or (Fab′)₂ fragment, or a genetically engineered Fv fragment (see U.S. Pat. No. 4,946,788, hereby incorporated by reference). In another aspect, the invention provides a method of detecting a leporipox virus polypeptide in a sample, by contacting the sample with the antibody of the invention, and assaying for the binding of the antibody to the polypeptide.

In another aspect, the invention provides a probe for analyzing a leporipox virus gene or a leporipox virus gene homolog or fragment thereof, the probe having at least 50% nucleotide sequence identity to a sequence encoding a leporipox virus polypeptide or fragment thereof, where the fragment includes at least six amino acids, and the probe hybridizes under high stringency conditions to at least a portion of a leporipox virus nucleic acid molecule. In a preferred embodiment of this aspect, the probe has 100% complementarity to a nucleic acid molecule encoding a leporipox virus polypeptide or fragment thereof, where the fragment includes at least six amino acids, and the probe hybridizes under high stringency conditions to at least a portion of a leporipox virus nucleic acid molecule.

In another aspect, the invention provides a method of detecting a leporipox virus gene or a leporipox virus gene homolog or fragment thereof in a cell, by contacting a leporipox virus nucleic acid molecule or a fragment thereof, where the fragment is greater than about 18 nucleotides in length, with a preparation of genomic DNA from said cell, under hybridization conditions providing detection of DNA sequences having about 50% or greater nucleotide sequence identity to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

In another aspect, the invention provides a method of identifying a leporipox virus gene or a leporipox virus gene homolog or fragment thereof by providing a mammalian cell sample; introducing by transformation into the cell sample a candidate gene; expressing the candidate gene within the cell sample; and determining whether the sample elicits an altered level of immune function, where an alteration in the level of immune function identifies a leporipox virus gene or a leporipox virus gene homolog or fragment thereof.

In another aspect, the invention provides a method of identifying a test compound that modulates the expression or activity of a leporipox virus polypeptide, by contacting said leporipox virus polypeptide with the test compound, and determining the effect of the test compound on the leporipox virus polypeptide.

In another aspect, the invention provides a method of targeting proteins for secretion from a cell, by attaching an identifiable signal sequence selected from a leporipox virus polypeptide to a protein of interest, such that protein of interest is secreted from said cell.

In another aspect, the invention provides a method of immunomodulation in a mammal, by administering to the mammal a therapeutically effective amount of a leporipox virus polypeptide or fragment thereof, where the polypeptide has an immunomodulatory effect in the mammal. In another aspect, the invention features a method of immunomodulation wherein the method involves: (a) providing the leporipox virus gene under the control of a promoter providing controllable expression of the leporipox virus gene in a cell wherein the leporipox virus gene is expressed in a construct capable of delivering a leporipox protein in an amount effective to decrease inflammation or inhibit autoimmune reaction. The polypeptide may also be provided directly, for example, in cell culture, for therapeutic uses. In preferred embodiments, leporipox virus polypeptide is delivered by expression of the leporipox virus gene using a tissue-specific or cell type-specific promoter, or by a promoter that is activated by the introduction of an external signal or agent, such as a chemical signal or agent. In preferred embodiments, the method is used for improving prognosis in patients with tumors. The method includes providing a leporipox virus protein that acts as an inducer of apoptosis in the region of the tumor either by providing an effective amount of the polypeptide or by providing an effective amount of a transgene expressing the polypeptide. In one such embodiment the tumor is a solid tumor, e.g. lymphoma (e.g., Hodgkin's), plasmacytoma, carcinoma (e.g., gastric, colonic, and lung carcinomas), melanoma, and sarcoma.

In another aspect, the invention features a recombinant polypeptide capable of mediating immunomodulatory events wherein the polypeptide includes a domain having a sequence which has at least 80% identity to at least one of the sequences of FIGS. 21-40. More preferably, the region of identity is 90% or greater; most preferably the region of identity is 95% or greater.

In another aspect, the invention provides a method of immunomodulation in a mammal, by administering to said mammal a therapeutically effective amount of a compound that modulates the activity of a leporipox virus polypeptide, where the compound has an immunomodulatory effect in said mammal.

In another aspect, the invention provides a method of treating a mammal having an immunomodulatory disorder, by administering to the mammal a therapeutically effective amount of a compound that modulates the activity of a leporipox virus polypeptide, where the compound has an immunomodulatory effect in said mammal. In other aspects, the invention provides a method for modulating cell (e.g., a mammalian cell) proliferation or apoptosis by administering to the cell a leporipox virus polypeptide or a compound that modulates the activity of a leporipox virus polypeptide (e.g., a chemical, a drug, or an antibody that specifically binds the leporipox virus polypeptide and neutralizes the polypeptide's activity). In related aspects, the present invention provides a method for treating AIDS, cirrhosis of the liver, neurodegeneration, myelodysplastic syndrome or ischemic injury by administration of a leporipox virus polypeptide with apoptotic activity.

In another aspect, the invention provides a pharmaceutical composition including at least one dose of a therapeutically effective amount of a leporipox virus polypeptide or fragment thereof, in a pharmaceutically acceptable carrier, the composition being formulated for the treatment of an immunomodulatory disorder.

In preferred embodiments of some of the above aspects, the leporipox virus polypeptide includes an amino acid sequence substantially identical to an amino acid sequence selected from the group consisting of SEQ ID NO:21, SEQ ID NO:22, SEQ ID NO:23, SEQ ID NO:24, SEQ ID NO:25, SEQ ID NO:26 or SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, and SEQ ID NO:40, and fragments and analogs thereof.

In other preferred embodiments of some of the above aspects, the immunomodulation includes immunosuppression, immunostimulation, cell proliferation, apoptosis, decreasing T cell stimulation, or decreasing inflammation in a mammal (e.g., a human). The mammal may be diagnosed with a tumor (e.g., a carcinoma, a plasmacytoma, a lymphoma, or a sarcoma).

In other preferred embodiments of some of the above aspects, the leporipox virus polypeptide is a multi-transmembrane receptor related protein or includes an amino acid sequence substantially identical to the sequence selected from the group consisting of SEQ ID NO:38, SEQ ID NO:39, and SEQ ID NO:40 and fragments and analogs thereof.

In other preferred embodiments of some of the above aspects, the mammal has a condition selected from the group consisting of acute inflammation, rheumatoid arthritis, transplant rejection, restenosis, asthma, allergies, inflammatory bowel disease, uveitis, psoriasis, atopic dermatitis, bronchial asthma, pollinosis, systemic lupus erythematosus, nephrotic syndrome lupus, multiple sclerosis, myasthenia gravis, type I and type II diabetes mellitus, glomerulonephritis, Hashimoto's thyroiditis, autoimmune hemolytic anemia, autoimmune thrombocytopenic purpura, microbial infection, malignancy and metastasis, autoimmune disease, cirrhosis, endotoxemia, atherosclerosis, reperfusion injury and inflammatory responses, AIDS, cirrhosis of the liver, neurodegeneration, myelodysplastic syndrome, and ischemic injury. It is particularly preferred that the mammal be diagnosed with an inflammatory or autoimmune disease (as those listed herein above). Alternatively, in other preferred embodiments, the mammal has a tumor (e.g., a carcinoma, a plasmacytoma, a lymphoma or a sarcoma).

In another aspect, the invention features a method of reducing inflammation. For example, damage occurring during asthmatic reactions, inflammatory bowel diseases (i.e., Crohn's Disease and ulcerative colitis), eosinophilic colitis, or allergic rhinitis. The method includes inhibiting inflammation normally caused by an immunostimulatory leporipox virus polypeptide. Preferably, leporipox virus polypeptide activity is reduced using an agonist such as an anti-leporipox virus polypeptide antibody or leporipox virus polypeptide fragment. In some embodiments, the antagonist is a leporipox polypeptide having a deletion of amino acids or having an addition of amino acids on the carboxy or alternatively the amino terminus. Where amino acids are added they may be random or they may be selected to have particular biological properties such as stability or hydrophilicity.

In another aspect, the invention features a method of isolating a leporipox virus gene or fragment thereof from a cell, involving: (a) providing a sample of cellular DNA; (b) providing a pair of oligonucleotides having sequence homology to a conserved region of a leporipox virus gene (for example, oligonucleotides which include fragments of the sequences shown in FIGS. 1-20); (c) combining the pair of oligonucleotides with the cellular DNA sample under conditions suitable for polymerase chain reaction-mediated DNA amplification; and (d) isolating the amplified leporipox virus gene or fragment thereof. Where a fragment is obtained by PCR, standard library screening techniques may be used to obtain the complete coding sequence. In preferred embodiments, amplification is carried out using a reverse-transcription polymerase chain reaction, for example, the RACE method.

In another aspect, the invention features a method of identifying a leporipox virus gene in a cell, involving: (a) providing a preparation of cellular DNA (for example, from the human genome); (b) providing a detectably-labeled DNA sequence (for example, prepared by the methods of the invention) having homology to a conserved region of a leporipox virus gene; (c) contacting the preparation of cellular DNA with the detectably-labeled DNA sequence under hybridization conditions providing detection of genes having 50% or greater sequence identity; and (d) identifying a leporipox virus gene by its association with the detectable label.

In another aspect, the invention features a method of isolating a leporipox virus gene from a recombinant DNA library, involving: (a) providing a recombinant DNA library; (b) contacting the recombinant DNA library with a detectably-labeled gene fragment produced according to the PCR method of the invention under hybridization conditions providing detection of genes having 50% or greater sequence identity; and (c) isolating a leporipox virus gene by its association with the detectable label.

In another aspect, the invention features a method of isolating a leporipox virus gene from a recombinant DNA library, involving: (a) providing a recombinant DNA library; (b) contacting the recombinant DNA library with a detectably-labeled leporipox virus oligonucleotide of the invention under hybridization conditions providing detection of genes having 50% or greater sequence identity; and (c) isolating a leporipox virus gene by its association with the detectable label.

In another aspect, the invention features a leporipox virus gene isolated according to the method involving: (a) providing a sample of cellular DNA; (b) providing a pair of oligonucleotides having sequence homology to a conserved region of a leporipox virus gene; (c) combining the pair of oligonucleotides with the cellular DNA sample under conditions suitable for polymerase chain reaction-mediated DNA amplification; and (d) isolating the amplified leporipox virus gene or fragment thereof.

In another aspect, the invention features a leporipox virus gene isolated according to the method involving: (a) providing a preparation of cellular DNA; (b) providing a detectably-labeled DNA sequence having homology to a conserved region of a leporipox virus gene; (c) contacting the preparation of DNA with the detectably-labeled DNA sequence under hybridization conditions providing detection of genes having 50% or greater sequence identity; and (d) identifying a leporipox virus gene by its association with the detectable label.

In another aspect, the invention features a leporipox virus gene isolated according to the method involving: (a) providing a recombinant DNA library; (b) contacting the recombinant DNA library with a detectably-labeled leporipox virus gene fragment produced according to the method of the invention under hybridization conditions providing detection of genes having 50% or greater sequence identity; and (c) isolating a leporipox virus gene by its association with the detectable label.

In another aspect, the invention features a method of identifying a leporipox virus gene involving: (a) providing a mammalian cell sample; (b) introducing by transformation (e.g. viral, chemical, or mechanical transformation) into the cell sample a candidate leporipox virus gene (e.g., obtained from a cDNA expression library); (c) expressing the candidate leporipox virus gene within the cell sample or isolating the leporipox virus polypeptide from the tissue sample or protein isolated therefrom; and (d) determining whether the cell sample elicits immunomodulatory activity (e.g., an alteration in neutrophil chemotaxis, where a decrease in neutrophil specific chemotaxis identifies a leporipox virus gene).

In another aspect, the invention features a leporipox virus gene isolated according to the method involving: (a) providing a cell sample; (b) introducing by transformation into the cell sample a candidate leporipox virus gene; (c) expressing the candidate leporipox virus gene within the tissue sample; and (d) determining whether the tissue sample elicits a leporipox virus protein mediated response or decrease thereof, where a response identifies a leporipox virus gene.

In another aspect, the invention features a method of detecting a leporipox virus gene in a cell involving: (a) contacting the leporipox virus gene or a portion thereof greater than 9 nucleic acids, preferably greater than 18 nucleic acids in length with a preparation of genomic DNA from the cell under hybridization conditions providing detection of DNA sequences having about 50% or greater sequence identity to the conserved DNA sequences of FIGS. 1-20, or the sequences which are conserved among leporipox virus polypeptides relative to other proteins, as deduced from the polypeptide sequences provided in FIGS. 21-40 Preferably, the region of sequence identity used for hybridization is the region of 9 nucleic acids or more encoding the region of highest conservation between the sequences shown in any of FIGS. 1-20 or among homologs thereof.

In another aspect, the invention features a method of producing a leporipox polypeptide (viral, rodent or human) which involves: (a) providing a cell transformed with DNA encoding a leporipox virus polypeptide positioned for expression in the cell (for example, present on a plasmid or inserted in the genome of the cell); (b) culturing the transformed cell under conditions for expressing the DNA; and (c) isolating the leporipox polypeptide.

### Definitions

“Polypeptide” or “polypeptide fragment” means a chain of two or more amino acids, regardless of any post-translational modification (e.g., glycosylation or phosphorylation), constituting all or part of a naturally or non-naturally occurring polypeptide. By “post-translational modification” is meant any change to a polypeptide or polypeptide fragment during or after synthesis. Post-translational modifications can be produced naturally (such as during synthesis within a cell) or generated artificially (such as by recombinant or chemical means). A “protein” can be made up of one or more polypeptides.

“Leporipox virus immunomodulatory polypeptide” or “leporipox virus polypeptide” means a leporipox virus polypeptide having immunomodulatory activity that is substantially identical to any of the myxoma virus polypeptide sequences described in FIGS. 21-40, or a fragment thereof. Specifically excluded from this definition are myxoma virus CBPI and CBPII, and Shope Fibroma virus CBPI and CBPII (U.S. Pat. No. 5,834,419, PCT publication WO 96/33730, and PCT publication WO 97/44054). A leporipox virus polypeptide may also be defined as encoding a polypeptide with at least 50%, preferably at least 75%, more preferably at least 90%, and most preferably at least 95% of the biological activity, e.g., immunomodulatory or anti-inflammatory activity, compared to a reference leporipox virus polypeptide having an amino acid sequence substantially identical to those described in FIGS. 21-40. The term leporipox virus polypeptide includes homologs, e.g. human or murine homologs, of the sequences described herein.

By “biologically active fragment” is meant a polypeptide fragment of a leporipox virus polypeptide that exhibits immunomodulatory properties that are at least 30%, preferably at least 50%, more preferably at least 75%, and most preferably at least 100%, compared with the immunomodulatory properties of a full length leporipox virus polypeptide. By “analog” is meant any substitution, addition, or deletion in the amino acid sequence of a leporipox virus polypeptide that exhibits properties that are at least 30%, preferably at least 50%, more preferably at least 75%, and most preferably at least 100%, compared with the immunomodulatory properties of a leporipox virus polypeptide from which it is derived. Fragments and analogs can be generated using standard techniques, for example, solid phase peptide synthesis or polymerase chain reaction.

“Leporipox virus immunomodulatory nucleic acid molecule” or “leporipox virus nucleic acid molecule” means a nucleic acid molecule a nucleic acid molecule, such as a genomic DNA, cDNA, or RNA (e.g., mRNA) molecule, that encodes a polypeptide having the characteristics or biological activities of any leporipox virus polypeptide described herein, or a fragment thereof. A leporipox virus nucleic acid molecule has at least 30% nucleic acid sequence identity, preferably at least 50% nucleic acid sequence identity, more preferably at least 75% nucleic acid sequence identity, and most preferably at least 95% nucleic acid sequence identity to a reference leporipox virus nucleic acid molecule as described herein.

The term “identity” is used herein to describe the relationship of the sequence of a particular nucleic acid molecule or polypeptide to the sequence of a reference molecule of the same type. For example, if a polypeptide or nucleic acid molecule has the same amino acid or nucleotide residue at a given position, compared to a reference molecule to which it is aligned, there is said to be “identity” at that position. The level of sequence identity of a nucleic acid molecule or a polypeptide to a reference molecule is typically measured using sequence analysis software with the default parameters specified therein, such as the introduction of gaps to achieve an optimal alignment (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). These software programs match identical or similar sequences by assigning degrees of identity to various substitutions, deletions, or other modifications. Conservative substitutions typically include substitutions within the following groups: glycine, alanine, valine, isoleucine, and leucine; aspartic acid, glutamic acid, asparagine, and glutamine; serine and threonine; lysine and arginine; and phenylalanine and tyrosine.

A nucleic acid molecule or polypeptide is said to be “substantially identical” to a reference molecule if it exhibits, over its entire length, at least 50%, preferably at least 55%, 60%, or 65%, and most preferably 75%, 85%, 90%, 95%, or 99% identity to the sequence of the reference molecule. For polypeptides, the length of comparison sequences is at least 16 amino acids, preferably at least 20 amino acids, more preferably at least 25 amino acids, and most preferably at least 35 amino acids. For nucleic acid molecules, the length of comparison sequences is at least 50 nucleotides, preferably at least 60 nucleotides, more preferably at least 75 nucleotides, and most preferably at least 110 nucleotides.

Alternatively, or additionally, two nucleic acid sequences are “substantially identical” if they hybridize under high stringency conditions. By “high stringency conditions” is meant conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8× SSC, 0.2 M Tris-Cl, pH 7.6, 1× Denhardt's solution, 10% dextran sulfate, and 0.1% SDS, at a temperature of 42° C. (These are typical conditions for high stringency northern or Southern hybridizations.) High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually 16 nucleotides or longer for PCR or sequencing and 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference.

By “probe” or “primer” is meant a single-stranded DNA or RNA molecule of defined sequence that can base pair to a second DNA or RNA molecule that contains a complementary sequence (“target”). The stability of the resulting hybrid depends upon the extent of the base pairing that occurs. This stability is affected by parameters such as the degree of complementarity between the probe and target molecule, and the degree of stringency of the hybridization conditions. The degree of hybridization stringency is affected by parameters such as the temperature, salt concentration, and concentration of organic molecules, such as formamide, and is determined by methods that are well known to those skilled in the art. Probes or primers specific for leporipox virus nucleic acid molecules, preferably, have greater than 45% sequence identity, more preferably at least 55-75% sequence identity, still more preferably at least 75-85% sequence identity, yet more preferably at least 85-99% sequence identity, and most preferably 100% sequence identity to the nucleic acid sequences encoding the amino acid sequences described herein Probes can be detectably-labeled, either radioactively or non-radioactively, by methods that are well-known to those skilled in the art. Probes can be used for methods involving nucleic acid hybridization, such as nucleic acid sequencing, nucleic acid amplification by the polymerase chain reaction, single stranded conformational polymorphism (SSCP) analysis, restriction fragment polymorphism (RFLP) analysis, Southern hybridization, northern hybridization, in situ hybridization, electrophoretic mobility shift assay (EMSA), and other methods that are well known to those skilled in the art. A molecule, e.g., an oligonucleotide probe or primer, a gene or fragment thereof, a cDNA molecule, a polypeptide, or an antibody, can be said to be “detectably-labeled” if it is marked in such a way that its presence can be directly identified in a sample. Methods for detectably-labeling molecules are well known in the art and include, without limitation, radioactive labeling (e.g., with an isotope, such as ³²P or ³⁵S) and nonradioactive labeling (e.g., with a fluorescent label, such as fluorescein).

By a “substantially pure polypeptide” is meant a polypeptide (or a fragment thereof) that has been separated from proteins and organic molecules that naturally accompany it. Typically, a polypeptide is substantially pure when it is at least 60%, by weight, free from the proteins and naturally-occurring organic molecules with which it is naturally associated. Preferably, the polypeptide is a leporipox virus polypeptide that is at least 75%, more preferably at least 90%, and most preferably at least 99%, by weight, pure. A substantially pure leporipox virus polypeptide can be obtained, for example, by extraction from a natural source (e.g., a mammalian cell), by expression of a recombinant nucleic acid molecule encoding a leporipox virus polypeptide, or by chemical synthesis. Purity can be measured by any appropriate method, e.g., by column chromatography, polyacrylamide gel electrophoresis, or HPLC analysis.

A polypeptide is substantially free of naturally associated components when it is separated from those proteins and organic molecules that accompany it in its natural state. Thus, a protein that is chemically synthesized or produced in a cellular system different from the cell in which it is naturally produced is substantially free from its naturally associated components. Accordingly, substantially pure polypeptides not only include those derived from eukaryotic organisms, but also those synthesized in E. coli or other prokaryotes.

An antibody is said to “specifically bind” to a polypeptide if it recognizes and binds to the polypeptide (e.g., a leporipox virus polypeptide), but does not substantially recognize and bind to other molecules (e.g., non-leporipox virus related polypeptides) in a sample, e.g., a biological sample, that naturally includes the polypeptide. A preferred antibody binds to any leporipox virus polypeptide sequence that is substantially identical to the polypeptide sequences shown in FIGS. 21-40, or portions thereof.

“Substantially pure nucleic acid molecule” means a nucleic acid molecule that is free of the components that naturally accompany it. For example, a substantially pure DNA is free of the genes that, in the naturally-occurring genome of the organism from which the DNA of the invention is derived, flank the gene. The term therefore includes, for example, a recombinant DNA which is incorporated into a vector; into an autonomously replicating plasmid or virus; or into the genomic DNA of a prokaryote or eukaryote; or that exists as a separate molecule (e.g., a cDNA or a genomic or cDNA fragment produced by PCR or restriction endonuclease digestion) independent of other sequences. It also includes a recombinant DNA that is part of a hybrid gene encoding additional polypeptide sequence.

By a “promoter” is meant a minimal nucleic acid sequence element sufficient to direct transcription. If desired, constructs of the invention can include promoter elements that are sufficient to render promoter-dependent gene expression controllable in a cell type-specific, tissue-specific, or temporal-specific manner, or inducible by external signals or agents. Such elements can be located in the 5′, 3′, or intron regions of a gene.

By “operably linked” is meant that a gene and one or more regulatory sequences are connected in such a way as to permit gene expression when the appropriate molecules (e.g., transcriptional activator proteins) are bound to the regulatory sequences.

By an “antisense molecule,” as used herein in reference to nucleic acid molecules, is meant a nucleic acid molecule having a sequence that is complementary to at least 75 nucleotides, and preferably at least 100, 150, or 200 nucleotides, of the coding strand of a gene, such as a leporipox virus gene. An antisense nucleic acid molecule can be, for example, capable of preferentially lowering the production of a leporipox virus polypeptide encoded by a leporipox virus gene.

By a “transgene” is meant a DNA molecule that is inserted by artifice into a cell (e.g., the nuclear genome of a cell), and is incorporated into the genome of an organism that develops from the cell. Such a transgene can be partly or entirely heterologous (i.e., foreign) to the transgenic organism, or can be a gene that is homologous to an endogenous gene of the organism. An organism or animal (e.g., a mammal, such as a mouse, rat, or goat) can be said to be “transgenic” if it developed from a cell that had a transgene inserted into it by artifice.

By a “knockout mutation” is meant an artificially-induced alteration in a nucleic acid molecule (created by recombinant DNA technology or deliberate exposure to a mutagen) that reduces the biological activity of the polypeptide normally encoded therefrom by at least 80% relative to the unmutated gene. The mutation can be, without limitation, an insertion, deletion, frameshift mutation, or a missense mutation. A “knockout animal” is preferably a mammal, and more preferably a mouse, containing a knockout mutation, as defined above.

By “vector” is meant a genetically engineered plasmid or virus, derived from, for example, a bacteriophage, adenovirus, retrovirus, poxvirus, herpesvirus, or artificial chromosome, that is used to transfer a polypeptide (e.g., a leporipox virus polypeptide) coding sequence, operably linked to a promoter, into a host cell, such that the encoded peptide or polypeptide is expressed within the host cell.

“Conserved region” means any stretch of six or more contiguous amino acids exhibiting at least 80%, preferably 90%, and most preferably 95% amino acid sequence identity between two or more of the leporipox virus family members.

By “transformation” is meant any method for introducing foreign molecules (e.g., nucleic acid molecules) into a cell. Lipofection, DEAE-dextran-mediated transfection, microinjection, protoplast fusion, calcium phosphate precipitation, retroviral delivery, electroporation, and biolistic transformation are just a few of the many transformation methods that are well known to those skilled in the art that can be used in the invention. For example, biolistic transformation is a method for introducing foreign molecules into a cell using velocity-driven microprojectiles such as tungsten or gold particles. Such methods can include helium-driven, air-driven, and gunpowder-driven techniques. Biolistic transformation can be applied to the transformation or transfection of a wide variety of cell types, intracellular organelles, and intact tissues including, without limitation, mitochondria, chloroplasts, bacteria, yeast, fungi, algae, animal tissue, and cultured cells. A “transformed cell,” “transfected cell,” or “transduced cell,” is a cell (or a descendent of a cell) into which a DNA molecule encoding a polypeptide has been introduced, by means of recombinant DNA techniques.

By “sample” is meant a tissue biopsy, amniotic fluid, cell, blood, serum, urine, stool, or other specimen obtained from a patient or test subject. The sample can be analyzed to detect a mutation in a leporipox virus gene, expression levels of a leporipox virus gene or polypeptide, or the biological function of a leporipox virus polypeptide, by methods that are known in the art. For example, methods such as sequencing, single-strand conformational polymorphism (SSCP) analysis, or restriction fragment length polymorphism (RFLP) analysis of PCR products derived from a patient sample can be used to detect a mutation in a leporipox virus gene; ELISA can be used to measure levels of leporipox virus polypeptide; and PCR can be used to measure the level of a leporipox virus nucleic acid molecule.

By “neutralizing antibody” is meant an antibody that interferes with any of the biological activities of a leporipox virus polypeptide, (e.g., the ability of the leporipox virus polypeptide M141 (mVOX2) to inhibit T cell costimulatory signals). A neutralizing antibody may reduce the ability of a leporipox virus polypeptide to carry out its specific biological activity by about 50%, more preferably by about 70%, and most preferably by about 90% or more. Any standard assay for the biological activity of any leporipox virus polypeptide, including those described herein, may be used to assess potentially neutralizing antibodies that are specific for leporipox virus polypeptides.

By “pharmaceutically acceptable carrier” is meant a carrier that is physiologically acceptable to the treated mammal while retaining the therapeutic properties of the compound with which it is administered. One exemplary pharmaceutically acceptable carrier is physiological saline solution. Other physiologically acceptable carriers and their formulations are known to one skilled in the art and described, for example, in Remington: The Science and Practice of Pharmacy, (19^{th} edition), ed. A. Gennaro, 1995, Mack Publishing Company, Easton, Pa.

By “test compound” is meant a chemical, be it naturally-occurring or artificially-derived, that is assayed for its ability to act as an immunomodulator, an anti-inflammatory, or an anti-tumor agent by employing one of the assay methods described herein. Test compounds may include, for example, peptides, polypeptides, synthesized organic molecules, naturally occurring organic molecules, nucleic acid molecules, and components thereof.

“Therapeutically effective amount” as used herein in reference to dosage of a medication, refers to the administration of a specific amount of a pharmacologically active agent (e.g., a leporipox virus polypeptide) tailored to each individual patient manifesting symptoms characteristic of a specific disorder. For example, a patient receiving the treatment of the present invention might be experiencing an autoimmune or inflammatory disease. A person skilled in the art will recognize that the optimal dose of a pharmaceutical agent to be administered will vary from one individual to another. Dosage in individual patients should take into account the patients height, weight, rate of absorption and metabolism of the medication in question, the stage of the disorder to be treated, and what other pharmacological agents are administered concurrently.

By “treating” or “treatment” is meant the medical management of a patient with the intent to cure, ameliorate, stabilize, or prevent a disease, pathological condition, or disorder. This term includes active treatment, that is, treatment directed specifically toward the improvement or associated with the cure of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. The phrase “treatment” also includes symptomatic treatment, that is, treatment directed toward constitutional symptoms of the associated disease, pathological condition, or disorder.

By “modulate” or “modulating” is meant changing, either by decrease or increase, the biological activity.

“Immune function” or “immunoreactivity” refers to the ability of the immune system to respond to foreign antigen as measured by standard assays well known in the art.

“Immunomodulation” or “immunomodulatory” refers to an alteration in the overall immunoreactivity of the immune system in a mammal upon treatment with an agent, such as a polypeptide of the present invention, or fragments and analogs thereof.

“Immunomodulator” refers to an agent that induces an alteration (i.e., immunosuppression or immunostimulation) as measured by an alteration of virulence in mutated viruses or a variety of immunoassays well known in the art (for example chemotaxis assays as described herein). For example, in the present invention, an immunomodulator may elicit an altered level of immune function whereby the alteration in the level of immune function identifies a leporipox virus polypeptide. Preferably, the alteration is by at least 20-40%, more preferably by at least 50-75%, and most preferably by more than 80% relative to an untreated control of similar type. By “immunomodulatory disorder” is meant any pathophysiological condition that is characterized by an alteration in immune function. The alteration may include, for example, a decrease in immune cell number or size, an increase in cell apoptosis or death, or a decrease in immune cell growth, survival or differentiation. By “immunomodulatory disorder” is also meant any disease which involves the immune response or immunity in general. More specifically, such a disorder is a malfunction of the immune system that reduces the ability of an organism to resist foreign substances in the body (e.g., viruses, bacteria, bacterial toxins, plant pollen, fungal spores, animal danders, medications, foods, allogeneic or xenogeneic transplanted organs) or causes the body to produce antibodies against its own tissues (e.g., autoimmune disorders), resulting in tissue injury. Immunological disorders can also occur when a malfunctioning immune system (caused by, for example, genetic defect, illness, injury, malnutrition, medications such as those -used for chemotherapy) results in an increase in frequency or severity of infections. Immunological disorders are often accompanied by inflammation, which is the body's reaction to tissue injury, and results in the accumulation of white blood cells, macrophages, and lymphocytes at the site of injury.

By “pathophysiological condition” is meant a disturbance of function and/or structure of a living organism, resulting from an external source, a genetic predisposition, a physical or chemical trauma, or a combination of the above, including, but not limited to, any mammalian disease.

“Immunosuppression” refers to a decrease in the overall immunoreactivity of the immune system upon administration of an immunomodulator in comparison to the immunoreactivity of an immune system that has not been contacted with the particular immunomodulator. Preferably, the decrease is by at least 20-40%, more preferably by at least 50-75%, and most preferably by more than 80% relative to an untreated control of similar type.

“Immunostimulation” refers to a increase in the overall immunoreactivity of the immune system upon administration of an immunomodulator in comparison to the immunoreactivity of an immune system that has not been contacted with the particular immunomodulator. Preferably, the increase is by at least 20-40%, more preferably by at least 50-75%, and most preferably by more than 80%.

“Decreasing T cell stimulation” means lowering the level of T cell stimulation as measured by, for example, chromium release assay, by at least 20-40%, more preferably by at least 50-75%, and most preferably by more than 80% relative to an untreated control of similar type.

“Decreasing inflammation” means decreasing the number of inflammatory cells (leukocytes, for example eosinophils) in the target tissue by at least 20-40%, more preferably by at least 50-75%, and most preferably by more than 80% relative to an untreated control tissue of similar type. Preferably the decrease in the number of leukocyte cells is at least two-fold.

By “cell proliferation” is meant the growth or reproduction of similar cells. By “inhibiting proliferation” is meant the decrease in the number of similar cells by at least 10%, more preferably by at least 20%, and most preferably by at least 50%. By “stimulating proliferation” is meant an increase in the number of similar cells by at least 10%, more preferably by at least 20%, and most preferably by at least 50%.

By “apoptosis” is meant the process of cell death where a dying cell displays a set of well-characterized biochemical hallmarks which include cytolemmal blebbing, cell soma shrinkage, chromatin condensation, and DNA laddering.

An “anti-inflammatory” agent is an immunomodulatory agent capable of decreasing the overall inflamation or immune function upon administration to an individual. Preferably, the decrease is by at least 20-40%, more preferably by at least 50-75%, and most preferably by more than 80% relative to an untreated control of similar type.

By “cytokine” is meant a small molecular weight polypeptide that plays an important role in regulating the immune response, by for example, signaling adjacent cells.

By “chemokine” is meant a small molecular weight ligand which is a chemoattractant for leukocytes (e.g., neutrophils, basophils, monocytes, and T cells), and is important for infiltration of lymphocytes and monocytes into sites of inflammation.

“Identifiable signal sequence” means a sequence of amino acids that may be identified by homology or biological activity to a peptide sequence with the known function of targeting a polypeptide to a particular region of the cell. Preferably the signal sequence directs the polypeptide to the cellular membrane wherefrom the polypeptide may be secreted. Alternatively, the signal sequence may direct the polypeptide to an intracellular compartment or organelle, such as the Golgi apparatus. One of ordinary skill in the art can identify a signal sequence by, for example, using readily available software (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs).

A “multi-transmembrane receptor protein” is an amphipathic protein having multiple hydrophobic regions that span the lipid bilayer of the cell membrane from the cytoplasm to the cell surface interspersed between hydrophilic regions that are exposed to water on both sides of the membrane. The number of hydrophobic regions in an amphipathic protein is often proportional to the number of times that proteins spans the lipid bilayer. Many multi-transmembrane receptor-related proteins act as receptors for various ligands (e.g., cytokines) that act to initiate transduction of a signal from the cell surface where the ligand is binding to the interior of the cell. The initiation is often in the form of a conformational change in the multi-transmembrane receptor upon ligand binding. One of ordinary skill in the art can such protein by, for example, using readily available software (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs).

Other features and advantages of the invention will be apparent from the following description of the preferred embodiments thereof, and from the claims.

### BRIEF DESCRIPTION OF THE DRAWINGS

FIG. 1 shows the cDNA sequence of M035 (SEQ ID NO:1).

FIG. 2 shows the cDNA sequence of M037 (SEQ ID NO:2).

FIG. 3 shows the cDNA sequence of M046 (SEQ ID NO:3).

FIG. 4 shows the cDNA sequence of M102 (SEQ ID NO:4).

FIG. 5 shows the cDNA sequence of M103 (SEQ ID NO:5).

FIG. 6 shows the cDNA sequence of M110 (SEQ ID NO:6).

FIG. 7 shows the cDNA sequence of M116 (SEQ ID NO:7).

FIG. 8 shows the cDNA sequence of M125 (SEQ ID NO:8).

FIG. 9 shows the cDNA sequence of M134 (SEQ ID NO:9).

FIG. 10 shows the cDNA sequence of M153 (SEQ ID NO:10).

FIG. 11 shows the cDNA sequence of M141 (mVOX-2) (SEQ ID NO:11).

FIG. 12 shows the cDNA sequence of M118 (SEQ ID NO:12).

FIG. 13 shows the cDNA sequence of M135 (SEQ ID NO:13).

FIG. 14 shows the cDNA sequence of M144 (SEQ ID NO:14).

FIG. 15 shows the cDNA sequence of M121 (SEQ ID NO:15).

FIG. 16 shows the cDNA sequence of M122 (SEQ ID NO:16).

FIG. 17 shows the cDNA sequence of M154 (SEQ ID NO:17).

FIG. 18 shows the cDNA sequence of M104 (SEQ ID NO:18).

FIG. 19 shows the cDNA sequence of M107 (SEQ ID NO:19).

FIG. 20 shows the cDNA sequence of M128 (SEQ ID NO:20).

FIG. 21 shows the amino acid sequence of M035 (SEQ ID NO:21).

FIG. 22 shows the amino acid sequence of M037 (SEQ ID NO:22).

FIG. 23 shows the amino acid sequence of M046 (SEQ ID NO:23).

FIG. 24 shows the amino acid sequence of M102 (SEQ ID NO:24).

FIG. 25 shows the amino acid sequence of M103 (SEQ ID NO:25).

FIG. 26 shows the amino acid sequence of M110 (SEQ ID NO:26).

FIG. 27 shows the amino acid sequence of M116 (SEQ ID NO:27).

FIG. 28 shows the amino acid sequence of M125 (SEQ ID NO:28).

FIG. 29 shows the amino acid sequence of M134 (SEQ ID NO:29).

FIG. 30 shows the amino acid sequence of M153 (SEQ ID NO:30).

FIG. 31 shows the amino acid sequence of M141 (mVOX-2) (SEQ ID NO:32).

FIG. 32 shows the amino acid sequence of M118 (SEQ ID NO:32).

FIG. 33 shows the amino acid sequence of M135 SEQ ID NO:33( ).

FIG. 34 shows the amino acid sequence of M144 (SEQ ID NO:34).

FIG. 35 shows the amino acid sequence of M121 (SEQ ID NO:35).

FIG. 36 shows the amino acid sequence of M122 (SEQ ID NO:36).

FIG. 37 shows the amino acid sequence of M154 (SEQ ID NO:37).

FIG. 38 shows the amino acid sequence of M104 (SEQ ID NO:38).

FIG. 39 shows the amino acid sequence of M107 (SEQ ID NO:39).

FIG. 40 shows the amino acid sequence of M128 (SEQ ID NO:40).

### DETAILED DESCRIPTION OF THE INVENTION

Typical naturally-occurring viral proteins act primarily as immunosuppressors.

Conceivably, these molecules have been obtained by the virus through an ancient act of molecular piracy and subsequently are structurally optimized for a particular pharmacological phenotype of benefit to the virus. Against the extensive array of immune modalities, viruses have successfully co-evolved distinct strategies to thwart the host immune response. The myxoma virus propagates in tissues (e.g., skin, respiratory tract and lymph nodes) that are readily accessible to a variety of the effector elements of the immune system. Like all poxviruses, myxoma has adapted to this inhospitable immune environment by expressing a wide range of proteins to systematically block each threatening aspect of the immune system.

The present invention provides novel genes that have the potential to treat a wide range of immunopathological conditions. Using standard molecular biological techniques well known in the art, a total of 1,925 DNA segments were isolated and sequenced using a shotgun sequencing approach with Bluescript primers (Stratagene, La Jolla, Calif.) to assemble a final contiguous myxoma viral gene sequence.

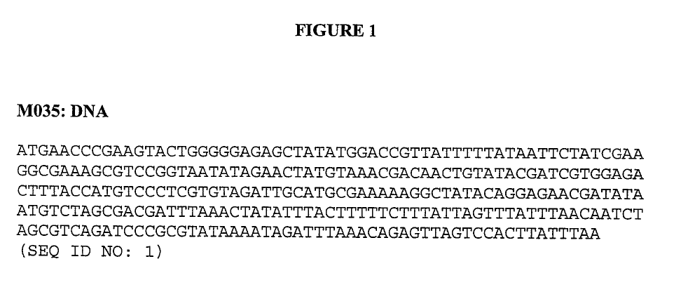

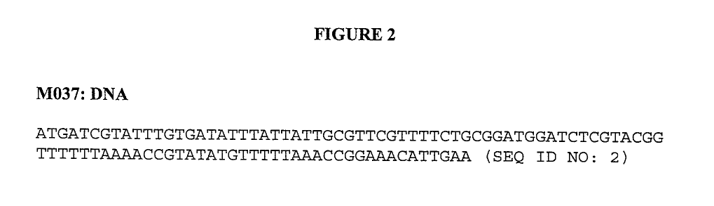

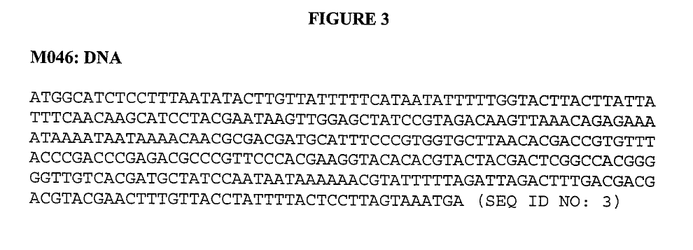

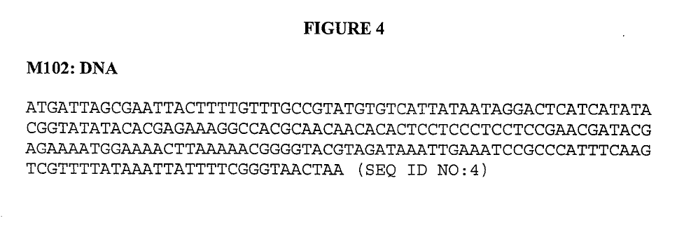

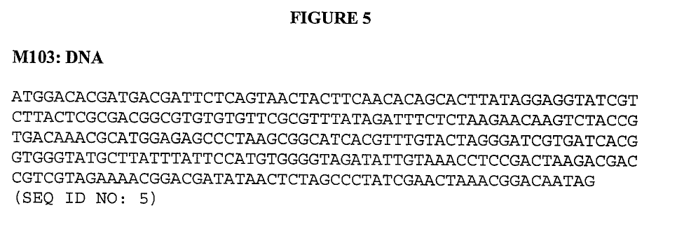

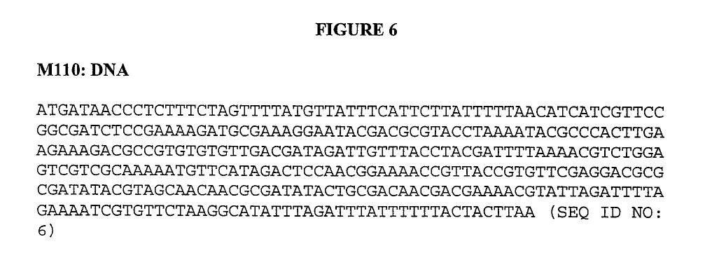

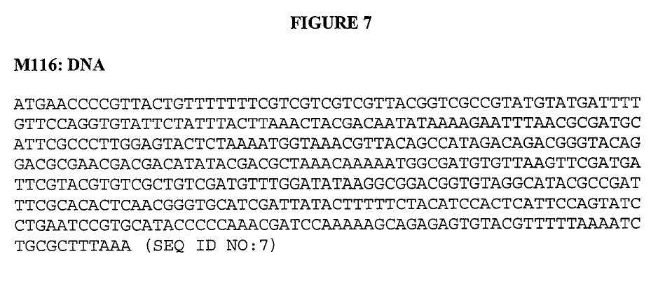

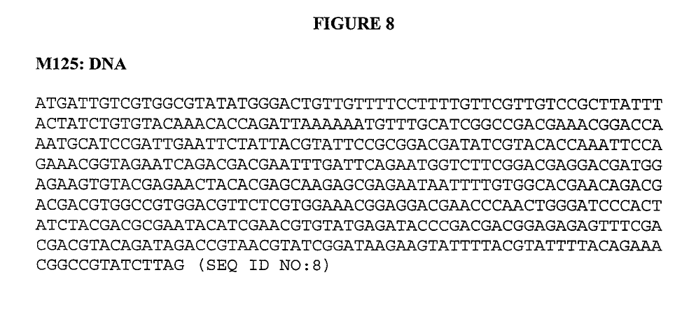

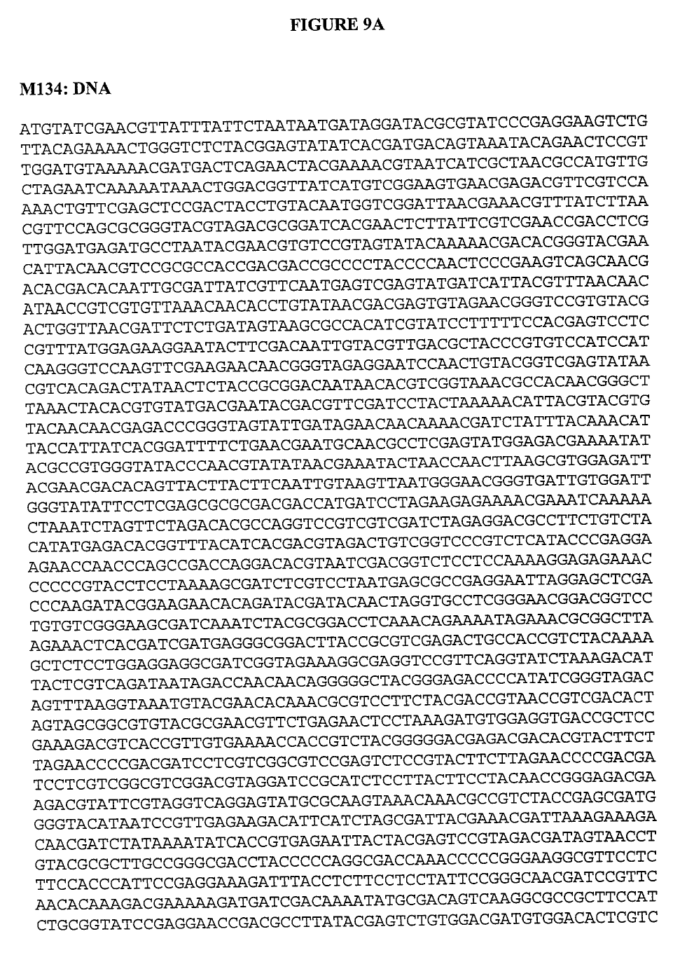

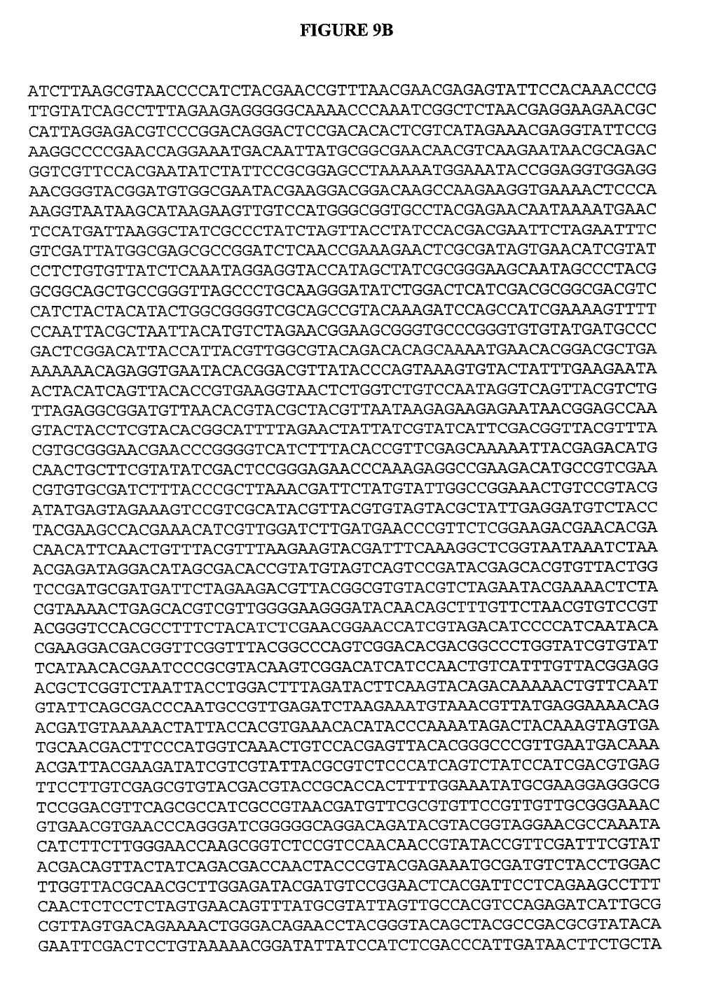

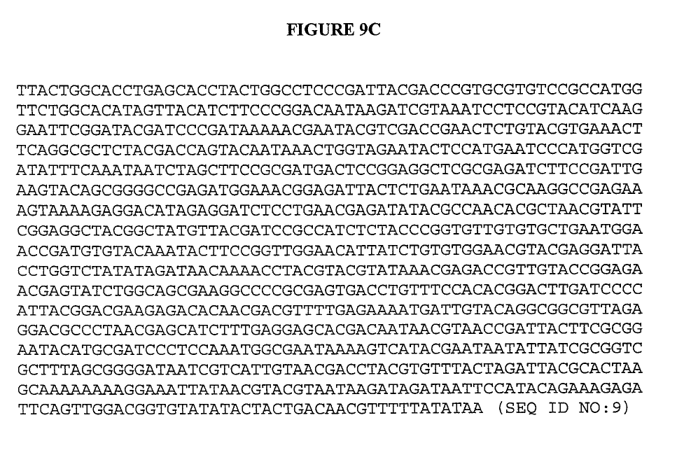

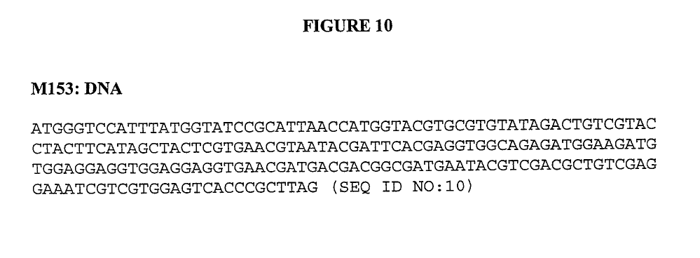

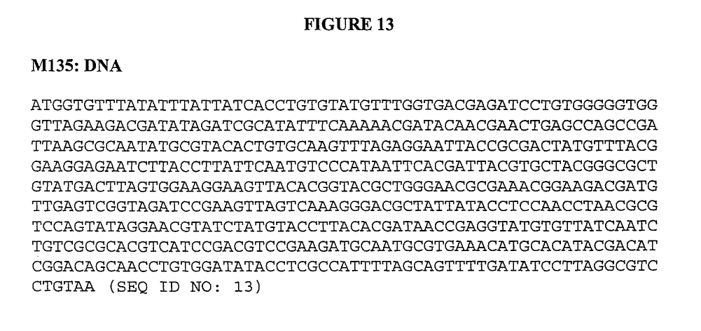

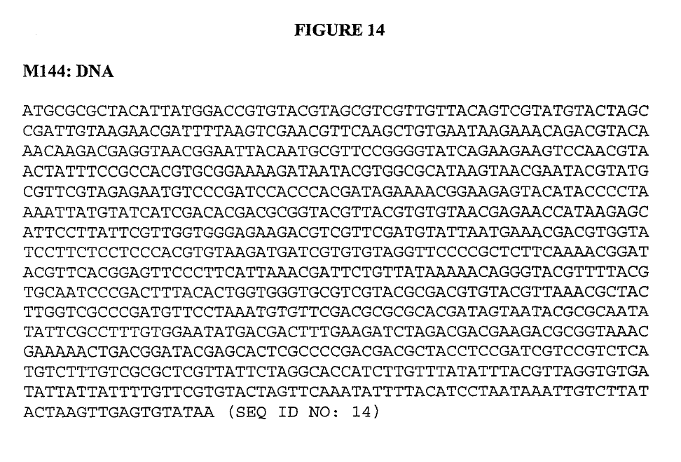

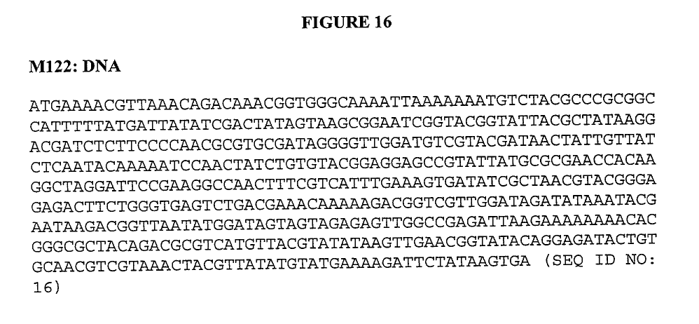

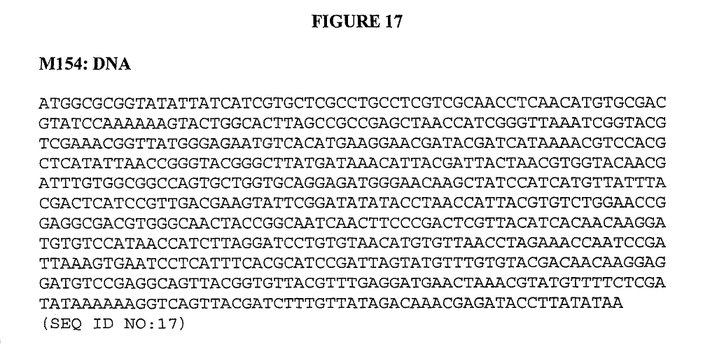

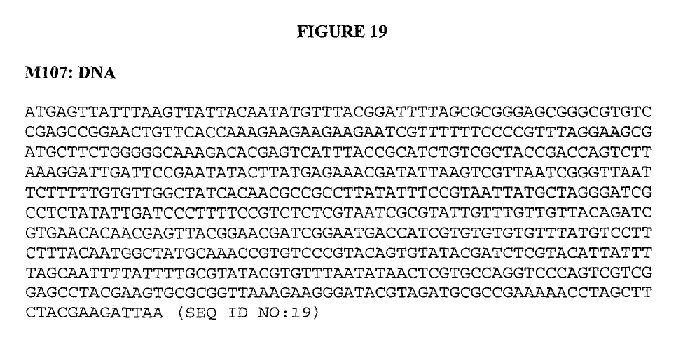

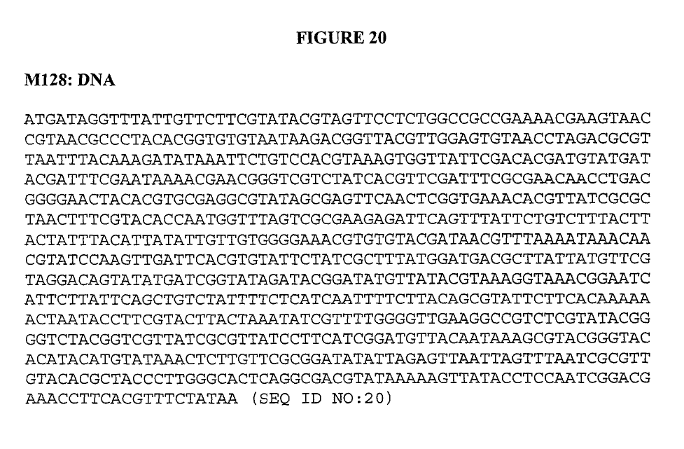

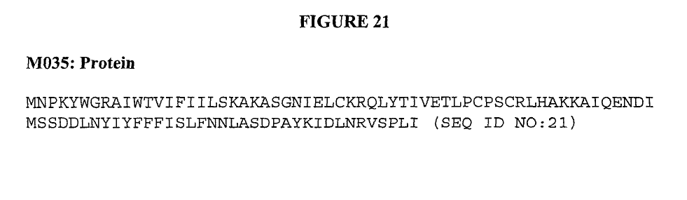

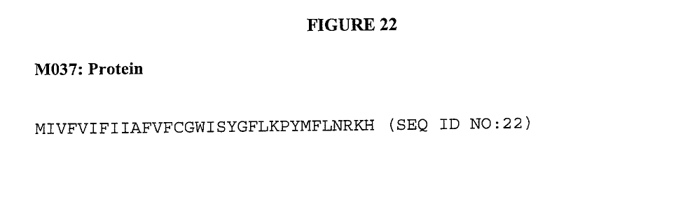

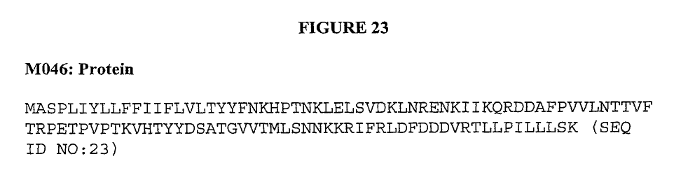

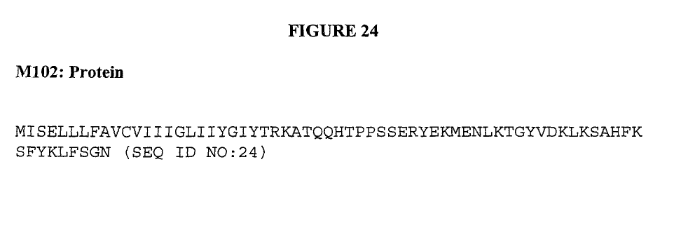

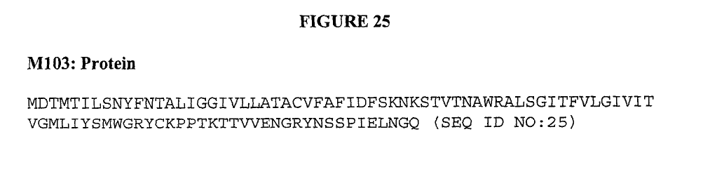

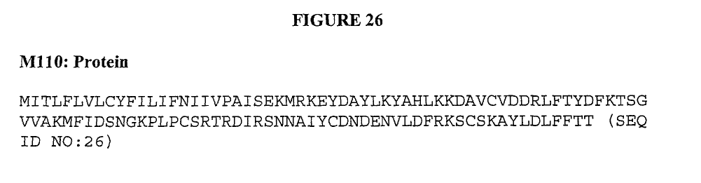

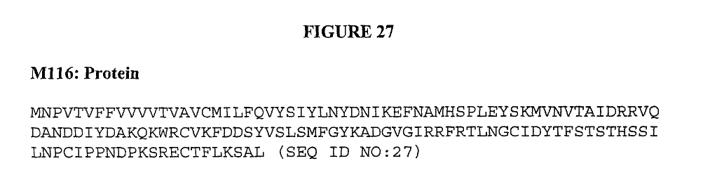

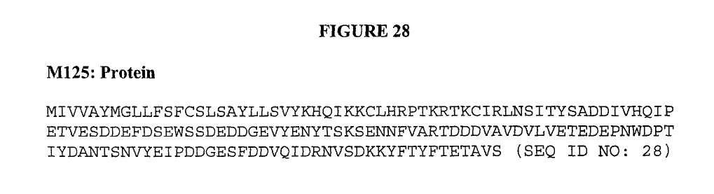

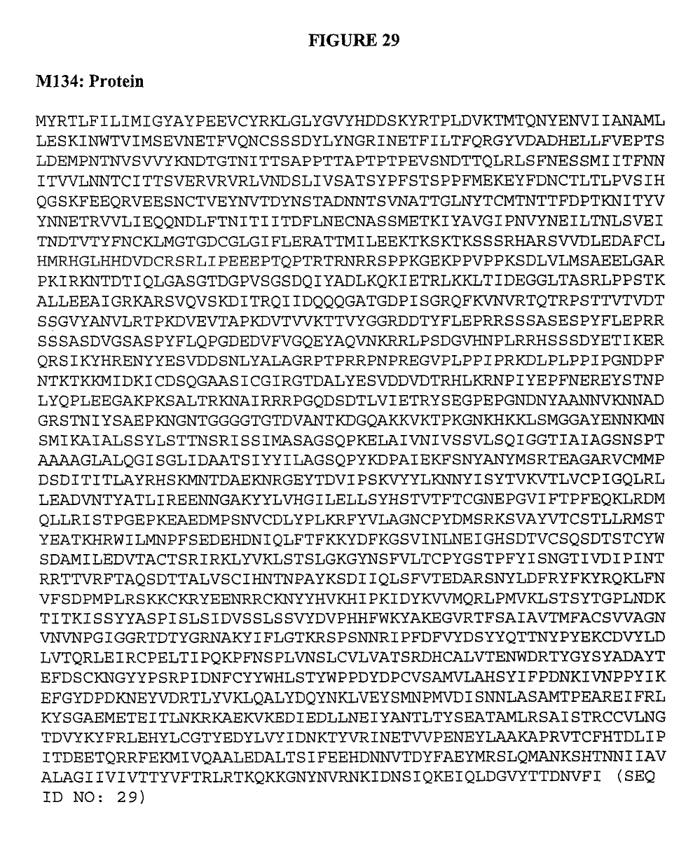

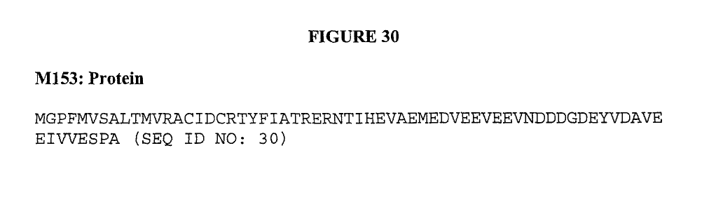

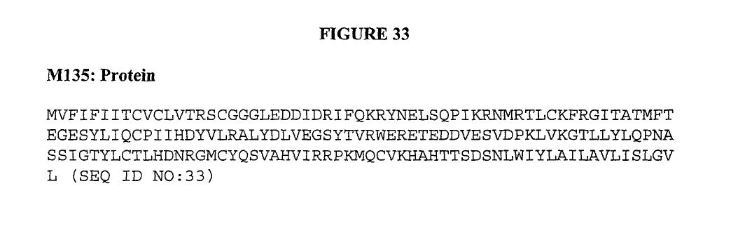

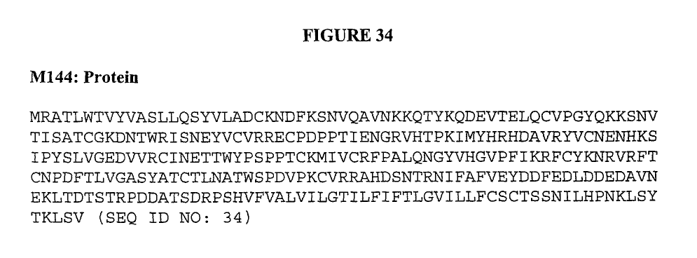

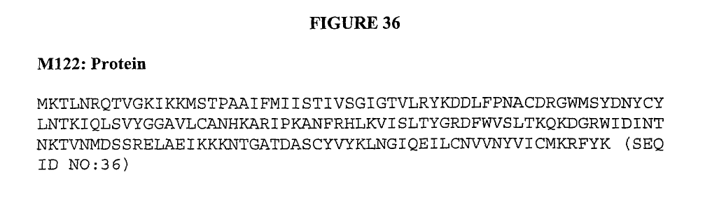

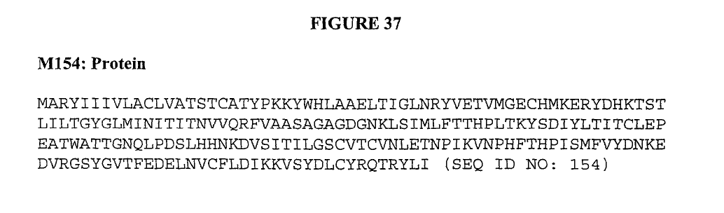

We have identified 20 novel genes within the myxoma genome that are herein disclosed as MO35, MO37, MO46, M102, M103, M110, M116, M125, M134, M153, M141, M118, M135, M144, M121, M122, M154, M104, M107, and M128 (SEQ ID NOs:1-20; FIGS. 1-20). The identification of start and stop codons within the cDNA sequences has permitted the prediction of the corresponding amino acid sequences (SEQ ID NOs: 21-40; FIGS. 21-40).

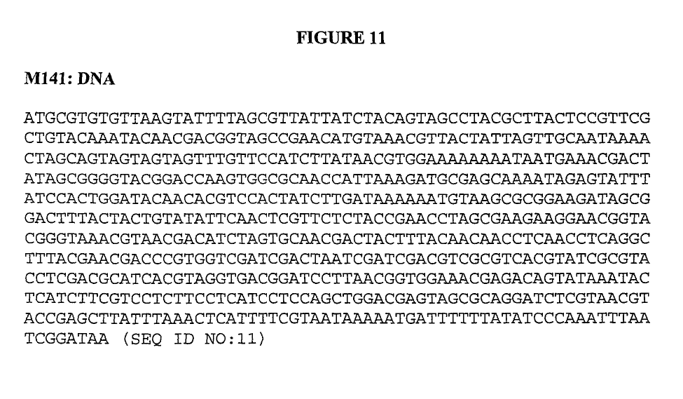

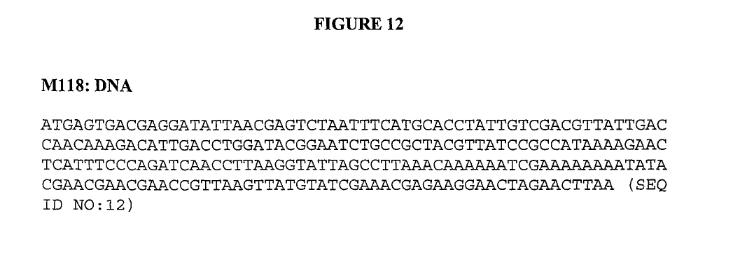

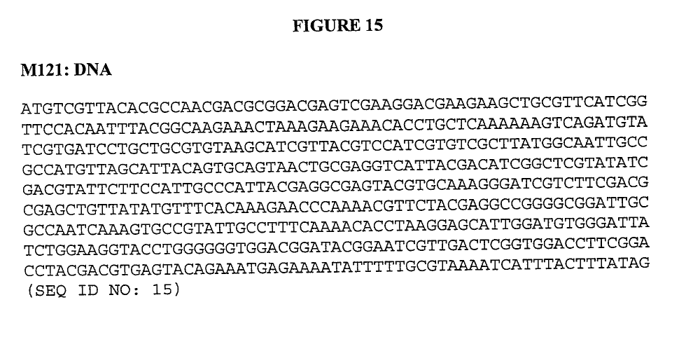

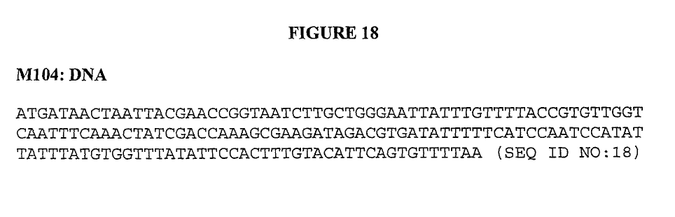

Many cDNA sequences of the present invention are novel, yet have significant homologies to other known genes or possess identifiable structural or functional domains. Predominantly, each of these genes encode polypeptides that are either homologous to an immunoregulatory protein or have recognizable domains that are known to have an immunoregulatory function. For example, cDNA sequence M141 (or mVOX) (SEQ ID NO:11; FIG. 11), has significant homology to the mammalian OX-2 gene. Some of the novel genes can be separated into three general categories: 1) anti-inflammatory proteins M118, M135, and M144 (SEQ ID NOs: 12-14; FIGS. 12-14, respectively); 2) immunoreceptor-regulated genes M121, M122, and M154 (SEQ ID NOs:15-17; FIGS. 15-17, respectively); and 3) multi-transmembrane receptor-related genes M104, M107, and M128 (SEQ ID NOs: 18-20; FIGS. 18-20, respectively).

The remaining novel genes encode proteins that are secreted from the myxoma-infected cells and have identifiable signal sequences. However, nucleic acid and protein sequence analysis algorithms have yielded no significant homology of these genes to known sequences. Furthermore, no structural or functional domains can be detected within the amino acid motif sequences disclosed herein. The functions of the proteins encoded by these novel genes may range among a wide variety of possibilities. Some of these genes may encode proteins that are important in modulating inflammation. Other genes may encode proteins that control viral replication and assembly. Alternatively, analysis of these genes and the polypeptides that they encode may elucidate the mechanism utilized by myxoma and possibly other leporipox viruses during infection and persistence within mammals. Still other proteins might be key regulators of cell proliferation or apoptosis. As mentioned previously, poxviruses express a variety of proteins that are able to modulate the innate cellular apoptotic response triggered by virus infection.

One of ordinary skill in the art will readily appreciate that any such proteins might be useful as therapeutic agents to treat a variety of human diseases. For example, the present invention may yield novel anti-inflammatories (e.g., for treatment of ischemic injury). Alternatively or additionally, the present invention may yield anti-viral compounds (e.g., for treatment of AIDS). Yet other proteins may be used as drugs for treatment of proliferative diseases (e.g., cancer or myelodysplastic syndrome) or cell death (e.g., neurodegeneration, muscular dystrophy, cirrhosis of the liver).

We believe that, upon expression in myxoma infected cells, for example, all of the genes disclosed herein, or modified variants thereof shall yield polypeptides that are secreted by the infected cell. This expectation is based upon two observations: 1) the absence of a retention sequence (such as the RDEL motif in T4) that would persistently localize these polypeptides to an organelle within the cell; and 2) the presence of a signal sequence that would mediate targeting of these proteins, either for insertion into the cell membrane or for secretion into the extracellular milieu. Some of the proteins of the present invention, i.e., those with significant homology to other known immunomodulatory proteins or functional domain are described in greater detail below.

Virokines

As mentioned above, one strategy of immune evasion by viruses is the capture and expression of host cytokines and growth factors (virokines). This strategy is employed by a number of viruses, which are restricted to the herpes virus and poxvirus families (McFadden et al., Semin. Cell. Dev. Biol., 9:359-368, 1996). Over time, the definition of virokine has expanded to encompass cytokine analogs and agonists, growth factors, and inhibitors, such as secreted serine proteinase inhibitors, of the poxvirus family (McFadden (1995) Austin (Tex.): R. G. Landes Company, incorporated herein by reference). Several sequences disclosed herein, including M141, M118, M135 and M144, may be classified as virokines. M135 and M144 can be more specifically identified as anti-inflammatories.

M141/mVOX-2

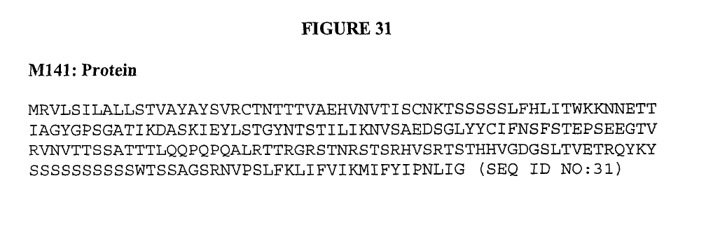

The cDNA sequence M141 (or mVOX) (SEQ ID NO:11) (see FIG. 11), has significant homology to the mammalian OX-2 gene. Specifically, M141 is predicted to encode a protein (SEQ ID NO:31; FIG. 31) having approximately 25% identity and 43% similarity to the human and rat isoforms OX-2. OX-2 is expressed in the brain, thymus, ovary and fallopian tubes, glomerulus, smooth muscle, as well as follicular dendritic cells in lymphoid organs, and the endothelium of the postcapillary venules (Borriello et al., J. Immunol. 158(10):4548-4554,1997). This widespread tissue distribution suggests a diverse role for OX-2. Previous studies have demonstrated that mammalian OX-2 receptors mediate costimulation of T cells in a B7-independent pathway. However, the mVOX-2 protein lacks a cytoplasmic domain that would presumably function in delivering intracellular signals.

It is currently thought that the immune response consists of two major components: a cellular immune response (mediated by T cells) and a humoral immune response (mediated by B cells). B cells recognize antigen via surface bound antibody that acts as a receptor. In contrast, T cells only recognize antigens through specific T cell receptors (TCR) expressed on the surface of T cells. TCR molecules only recognize linear epitopes that have been processed and presented onto the surface of an antigen presenting cell (APC). These linear epitopes have been processed intercellularly and presented on the surface of the APC in association with major histocompatibility complex MHC molecules.

Activation of T cells requires two distinct types of signals. The first signal is the antigen dependent signal and results from the engagement of the TCR by major histocompatibility (MHC)-antigen complex on the surface of the APC. The second signal is antigen independent, and is mediated by interaction of one of a number of ligands on APCs with their cognate receptor(s) on T cells. It is only when these two distinct signals are delivered together to the T cell, that T cell proliferation and cytokine production ensue. This important control point in T cell activation relies on the ligand B7 and the receptor CD28. The ligands of the B7/CD28 pathway (B7-1 and B7-2) are expressed on a variety of APCs and can provide costimulation through the CD28 receptor on T cells.

It has been demonstrated that OX-2 is also capable of providing a potent second signal to T cells by costimulation through OX-2 receptors on T cells (in the presence of antigen), (Borriello et al., J. Immunol., 158(10):4548-4554, 1997). Signaling through OX-2 results in T cell proliferation and antigen specific tolerance. OX-2 can also provide costimulation to T cells in an antigen-independent manner using an anti-CD3 antibody to provide the primary signal (Borriello et al., J. Immunol., 158(10):4548-4554, 1997). Furthermore, it has been shown that OX-2 costimulation prevents a Th1 type of immune response and thereby mediates transplant tolerance (Gorczynski et al., Transplantation, 65(8):1106-1114, 1999).

We believe that mVOX-2 expressed during myxoma infection can have one or both of the following effects: 1) suppression of the Th1 immune response needed for viral clearance and cytotoxic T lymphocyte activity; and 2) inhibition of T cell costimulatory signals due to the lack of an intracellular signaling domain. In this scenario, mVOX-2 acts in a dominant negative fashion. Therefore, mVOX-2 expression most likely plays an immunosuppressive role during myxoma infection of mammalian organisms.

In one aspect of the invention, the polypeptide encoded by M141, the mVOX-2 gene, is administered in vivo as an immunosuppressant. For example, in one preferred embodiment, a therapeutic preparation of mVOX-2 polypeptide can be used to modulate cytotoxic immune responses. Alternatively, in another preferred embodiment of the invention, administration of mVOX-2 in vivo disrupts T cell stimulation.

M118/Mig Homolog

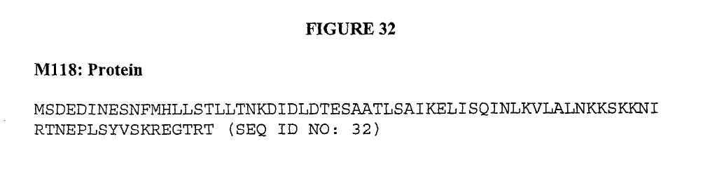

The DNA sequence referred to herein as M118 (SEQ ID NO:12) (see FIG. 12) has a protein sequence (SEQ ID NO:32) (see FIG. 32) that demonstrates considerable homology to a mammalian chemokine known as “monokine induced by interferon-g” (Mig). As mentioned above, chemokines are a large family of small (67 to 103 amino acids) secreted proteins that play an important role in the selective recruitment of leukocytes (Baggiolini et al., Annu. Rev. Immunol. 15:675-705, 1997). Generally, a chemokine is a chemotactic cytokine that ensures migration of leukocytes to the right tissue or compartment at the right time. Chemokines are rapidly produced in blood and tissue cells upon induction by pro-inflammatory cytokines and other stimuli (Baggiollni et al., supra; Moser et al., Int. Rev. Immunol., 16:323-344, 1997; Murphy, Cytokine Growth Fact. Rev. 7:47-64, 1996; Gerard et al., Curr. Opin. Immunol. 6:140-145, 1996). While the mammalian Mig contains two conserved cysteine residues separated by an amino acid (and hence is a member of the CXC family of chemokines), the myxoma M118 homolog does not retain such cysteines. Furthermore, M118 homology to Mig is limited to the C-terminal region of the protein.

Thousands of chemokine receptors are expressed on the surface of a single leukocyte. Upon ligand binding, leukocytes respond by chemotaxis, enzyme and mediator release and various effector functions. All known chemokine receptors are members of the large family of seven-transmembrane domain receptors that require G proteins for signal transduction. As mentioned previously, the chemokine receptors can be further classified into two distinct families, CC and CXC, based on which family of chemokines they bind (Baggiolini et al., supra). Based on this complexity, it will be apparent to one of ordinary skill in the art, that the cellular infiltrate is largely defined by the composition of locally produced chemokines as well as the diversity of circulating leukocytes that express the relevant receptors.

The amino acid sequence homology of M118 to mammalian Mig provides insight into its role during myxoma infection of mammalian hosts. Mig is known to be upregulated in response to secretion of interferon-g IFN-g (Luster et al., Nature 215:272-276, 1985; Farber, Proc. Natl. Acad. Sci. U.S.A. 87, 5238-5242, 1998; Farber, Biochem. Biophys. Res. Commun. 192:223-230,1993). Previous studies have demonstrated that mammalian Mig binds the CXC Receptor 3 (CXCR3) (Piali et al., Eur. J. Immunol. 28(3):961-972, 1998; Goebeler et al., J. Pathol., 184(1):89-95, 1998) and acts primarily as a potent chemoattractant on activated T and natural killer (NK)-cells (Luster et al., J. Exp. Med., 178:1057-1066, 1993; Farber, J. Leukocyte Biol. 61:246-257, 1997). Hence, it is suggested that Mig is involved in the recruitment of lymphocytes during inflammation and mediates the progression of interferon dependent pathologies. Lymphocyte participation is prominently associated with delayed-type hypersensitivity, autoimmune and antiviral responses. For example, it is likely that Mig is expressed during myxoma infection of mammalian cells. By way of further example, expression of Mig also correlates with psoriatic lesions (Weng et al., J. Biol. Chem., 272(29):18288-18291, 1998).

We believe that the M118 protein antagonizes the chemotactic function of Mig, since it only represents a C-terminal fragment of the mammalian Mig and does not contain the two cysteine residues conserved within the CXC subfamily of chemokines. A chemokine antagonist could be used by the virus to prevent the local recruitment of leukocytes. We believe that M118 may disrupt Mig function by blocking the Mig binding site upon CXCR3. Alternatively or additionally, M118 may otherwise abrogate CXCR-mediated signaling. As a result, M118 may provide an anti-inflammatory role during myxoma infection of mammalian organisms. We also propose that the in vivo administration of M118 gene products shall reduce Mig-CXCR3 mediated signaling and therefore decrease or interrupt lymphocyte recruitment and inflammatory symptoms associated with any of a variety of inflammatory diseases. Therefore, preferred embodiments of the present invention provide the polypeptide encoded by the M118 gene for administration in vivo as an immunomodulatory compound with anti-inflammatory effects.

M135 and M144/Anti-inflammatories

We believe that the M135 and M144 sequences disclosed herein will mediate anti-inflammatory mechanisms in vivo upon myxoma infection through the proteins encoded therein. The predicted amino acid sequences of M135 and M144 are homologous to important known mammalian anti-inflammatory genes.

Upon analysis, M135 appears to be a hybrid between the IL-1b and the IL-6 receptor. M135 shares 29% identity (47% similarity) with the known sequence of the IL-1b receptor and shares homology with the IL-6 receptor. In addition to the activity of activated T cells in mediating cellular immune responses, cellular immunity is also mediated by lymphocyte products, including interleukins and lymphokines, that are released by the T cells and other immunomodulatory cells (e.g., monocytes, dendritic cells, B cells, fibroblasts and epithelial cells) upon contact with antigen. Receptors for IL-1b transduce signals that regulate inflammations and are found on a number of different cells including, thymocytes, monocytes, T cells, B cells and monocytes. IL-6 responding cells include thymocytes, B cells and NK cells. The IL-6 receptor mediates signals that promote differentiation.

It is apparent from the diversity in function of the homologous proteins listed above that any of a variety of immunomodulatory activities could be affected by the gene products of the M135. For example, without wishing to be bound to any particular theory, M135 may function as a dominant negative IL-1b receptor that binds the IL-1 or IL-6 ligand and prevents intracellular signaling. In preferred embodiments, the invention provides a polypeptide encoded by M135 that can be administered in vivo as an immunomodulatory protein with immunosuppressant effects.

M144 shares 27% identity (42% similarity) with CD46, a known complement regulator that mediates the lysis of infected cells. As mentioned previously, a humoral immune response is carried out by B cells in their production of antibodies that specifically recognize a particular antigen. Although antibody alone may in some instances be protective in vivo, much of the ability of antibodies to protect against infection is dependent on the complement system. On reaction of an antibody with its antigens a collection of blood serum proteins (referred to herein as “complement”) are activated. As a result of this activation, organisms expressing a particular antigen may be lysed and in the process, phagocytic cells are called to remove the debris. As an example, lysis of antibody-coated bacteria may occur as a result of the complement-mediated pathway.

In addition to the antibody initiated sequence of complement events, there is an alternate pathway for the activation of complement components. This system involves a series of components that interact with polysaccharides and lipopolysaccharides on bacteria or virus particles, or with parasites. This alternate pathway for the activation of complement is important for control of infection in the absence of specific immunity. Thus, many different organisms are eliminated as a result of their activation of the alternate pathway.

Inappropriate or excessive activation of the complement system can lead to harmful, potentially life-threatening consequences due to severe inflammatory tissue destruction. These consequences are clinically manifested in various disorders, including septic shock, multiple organ failure, and hyper-acute graft rejection. Genetic complement deficiencies or complement depletion have been proven to be beneficial in reducing tissue injury in a number of animal models of severe complement-dependent inflammation. It is therefore believed that therapeutic inhibition of complement is likely to arrest the process of certain diseases.

Attempts to efficiently inhibit complement include the application of endogenous soluble complement inhibitors, the administration of antibodies, and either blockage of key proteins of the cascade reaction, neutralizing the action of the complement-derived anaphylatoxin, or interfering with complement receptor 3 (CR3, CD18/11b)-mediated adhesion of inflammatory cells to the vascular endothelium. In addition, incorporation of membrane-bound complement regulators (DAF-CD55, MCP-CD46, CD59) has become possible by transfection of the correspondent cDNA into xenogeneic cells. Protection against complement-mediated inflammatory tissue damage could be achieved in various animal models of sepsis, myocardial as well as intestinal ischemia/reperfusion injury, adult respiratory distress syndrome, nephritis, and graft rejection. Thus, complement inhibition appears to be a suitable therapeutic approach to control inflammation (Kirschfink et al., Immunopharmacology Dec;38(1-2):51-62, 1997).

Human CD46 has been identified as 1) a measles virus receptor protein; and 2) a regulator of the complement pathway. During measles virus (MV) infection, lymphopenia and immune suppression are observed in humans. CD46 (also known as membrane cofactor protein (MCP)) acts as a receptor for MV, accelerating entry of the virus into host cells. As a regulator of the complement cascade, CD46 acts to protect host cells from the autologous complement system. CD46, in addition to CD55 (decay-accelerating factor) and CD59 (protecting, has also been shown to protect tumor cells against lysis by activated complement (Schmitt et al., (1999) Eur. J. Cancer, Jan;35(l):117-24). Thus, CD46 encompasses both complement-related and MV-mediated immunomodulation (Seya, Int. J. Hematol. Aug;64(2):101-9, 1996; Devaux et al., Eur. J. Immunol., Mar;29(3):815-22, 1999).

We believe that M144 encodes a dominant negative form of CD46 that prevents the proper formation of the complement complex and lysis of infected cells. We believe that in vivo administration of the M144 gene product will reduce inflammatory symptoms. Alternatively, the M144 gene product may be employed to inhibit viral entry into cells.

Viroceptors

As mentioned above, cytokine receptors have been evolutionarily acquired by poxviruses. The term viroceptor includes secreted soluble virus-encoded cytokine receptors. Viroceptors function 1) by binding host cytokines, thereby preventing subsequent interactions with their true cellular receptors; and 2) as virus-encoded transmembrane proteins with significant homology to cellular cytokine receptors. Several of the sequences disclosed in the present invention can be classified as viroceptors, including M121, M122, M154, M104, M107, and M128, which are described in more detail below. M121, M122 and M154 are immunoreceptor-related genes, whereas M104, M107 and M128 are multi-transmembrane receptor-related genes.

Immunoreceptor-related Genes

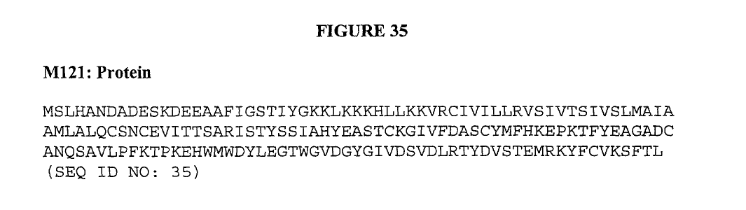

As previously mentioned, the DNA sequences M121, M122, and M154 (SEQ ID NO:15, SEQ ID NO:16 and SEQ ID NO:17) (see FIGS. 15-17) are predicted to encode immunoreceptor-related proteins (SEQ ID NO: 35, SEQ ID NO: 36 and SEQ ID NO:37) (see FIGS. 35-37) that are expressed by myxoma-infected cells. Immunoreceptors are key response molecules that receive and respond to signals embodied as receptor ligands. Immunoreceptors receive information from the extracellular milieu and transmit that information to the intracellular environment enabling coordination of the many complex -interactions required to modulate an immune response. It is the theory of the present invention that the proteins encoded by the novel myxoma genes M121, M122, and M154 function to interrupt the normal function of the homologous immunoreceptor proteins expressed by immune cells, thereby inhibiting immunostimulation.

The M121 protein demonstrates high homology (24% identity) to the CD69 protein, a C-type lectin receptor that mediates recognition of carbohydrate molecules. A lectin is a protein with binding sites that recognize specific sugar residues. Such specific protein-carbohydrate interactions serve multiple functions in the immune system. For example, many animal lectins mediate both pathogen recognition and cell-cell interactions. In addition, the broad selectivity of the sugar-binding site on a lectin contributes to the immune system's ability to discriminate between self and non-self (Weiss et al., Immunol Rev 1998 Jun;163:19-34, 1998). Specific C-type lectin receptors can distinguish amongst a wide variety of lectins. We believe that expression of M121 protein by myxoma-infected cells competes with the CD69 receptor for specific carbohydrate ligands and thereby interrupts CD69-mediated responses of immune cells.

In preferred embodiments, the present invention provides the polypeptide encoded by the M121 gene. In particularly preferred embodiments, the M121 polypeptide is administered in vivo as an immunosuppressant. For example, in one aspect embodiment, the present invention provides a therapeutic preparation of M121 polypeptide that may be used to modulate pathogen recognition. Alternatively, in another aspect of the invention, administration of M121 in vivo may promote discrimination between self and non-self.

The M122 protein also shares (22%) identity to a C-type lectin receptor, specifically LY49C (herein referred to as NKR), over a 127 amino acid region. NKR mediates natural killer (NK) cell immune responses (Accession # G11330631). The M122 protein may therefore be secreted by myxoma infected cells to disrupt the function of NK cells within the immune system and provide protection against an NK mediated inflammatory response. Therefore, in preferred embodiments, the present invention provides the M122 protein as a therapeutic immunosuppressant agent.

The M154 protein exhibits homology to other viral proteins, including the gp120 protein of the Human Immunodeficiency Virus (HIV) (21%). It is well known that HIV infects CD4+ T cells. The tropism of HIV for these cells is determined through the envelope protein gp120, which binds the CD4 receptor (along with additional co-receptor molecules) (Paxton et al., Semin. Immunol., Jun;10(3):187-194, 1998) and enables the virus to fuse with the cell membrane, thereby being taken up by the cell. Binding of gp120 to CD4 also plays a major role in downregulating CD4+ T cells, leading to the characteristic decline in the number of CD4+ T cells in HIV infected patients. One theory is that gp120 binding to CD4+ T cells plays a role in activating a cell death pathway in T cells (Peter et al., Br. Med. Bull, 53(3):604-16, 1997). Due to the known role of the gp120 protein in downregulating CD4+ T cells, we believe that in vivo expression of M154 binds and disrupts immunoreceptor function to persist myxoma infection. Thus, certain preferred embodiments of the invention include preparations of the M154 polypeptide that can be used as suppressors of immunoreceptor function.

Multi-transmembrane Receptors

As previously mentioned, the DNA sequences M104, M107, and M128 (SEQ ID NO:18, SEQ ID NO:19 and SEQ ID NO:20) (see FIGS. 18-20) are predicted to encode homologs of several mammalian receptor proteins that contain multiple transmembrane regions. Multi-transmembrane receptor proteins are amphipathic proteins having multiple hydrophobic regions that span the lipid bilayer of the cell membrane from the cytoplasm to the cell surface interspersed between hydrophilic regions that are exposed to water on both sides of the membrane. The number of hydrophobic regions in an amphipathic protein is often proportional to the number of times that proteins spans the lipid bilayer. Many multi-transmembrane receptor-related proteins act as receptors for various ligands (e.g., cytokines) that initiate transduction of a signal from the cell surface, where the ligand is binding, to the interior of the cell. This initiation is often in the form of a conformational change in the multi-transmembrane receptor upon ligand binding. Such complex formation is an important event in transducing immunomodulatory signals that are important in initiating or suppressing certain immune functions.

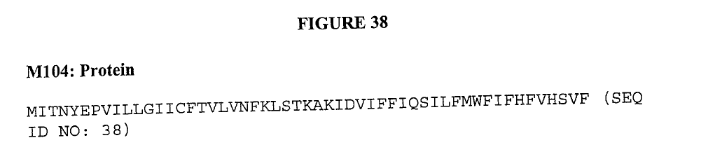

The M104 protein (SEQ ID NO: 38) (FIG. 38) is a short polypeptide containing high sequence similarity to the ORF-74 IL-8 receptor encoded by Ateline herpesvirus (Rosenkilde et al., J. Biol. Chem., Jan 6;274(2):956-961, 1999). ORR-74 is a CXC chemokine receptor encoded by many g-herpesviruses (e.g., human herpesvirus 8 and Kaposi's sarcoma-associated herpesvirus). ORF-74 is a seven transmembrane (7TM) protein that binds the chemokine IL-8 with high affinity, although it has limited homology to mammalian IL-8 receptors (CXCR-1 and CXCR-2).

7TM protein receptors frequently signal through G proteins (for reviews see Kelvin et al., J. Leukocyte Biol., 54:604-612, 1993; Murphy, Ann. Rev. Imm., 12:593-633, 1994; Horuk, Imm. Today, 15:169-174,1994). Some G proteins (Gₛ and Gᵢ) couple receptors to the adenylate cyclase cascade, whereas others (Gₚ) couple receptors to the phospholipase C signaling pathway. Most 7TM chemokine receptors preferentially signal through the Gᵢ pathway, however ORF-74 activates the phospholipase C pathway, leading to constitutively high turnover of various mitogenic proteins (e.g., phosphatidylinositol, cJun, N-terminal kinase, and p38 mitogen activated protein kinase). Therefore, ORF-74 also functions as an oncogene that induces cellular transformation. It has been proposed that ORF-74 could be causally involved in the development of Kaposi's sarcoma lesions and lymphomas associated with herpesvirus infection (Rosenkilde et al., supra).

While the wild type ORF74 protein consists of seven transmembrane regions, the M104 polypeptide disclosed herein only demonstrates one or two C-terminal transmembrane domain linked to a short cytosolic tail. Hence, without wishing to limit the theory of the invention, we suggest that upon expression of M104 by infected cells, M104 polypeptide molecules interfere with IL-8 mediated signal transduction and act as dominant negative versions of the IL-8 receptor or other chemokine receptors. Thus, in one aspect of the invention, M104 is used as an immunosuppressant. Given the potential role of ORF-74 in cellular transformation and malignancy, another aspect of the invention provides antagonists to ORF-74 that can be used as treatments for herpesvirus-associated malignancies. Such treatments are particularly desirable because it is well known that 7TM receptors are classically good drug targets.

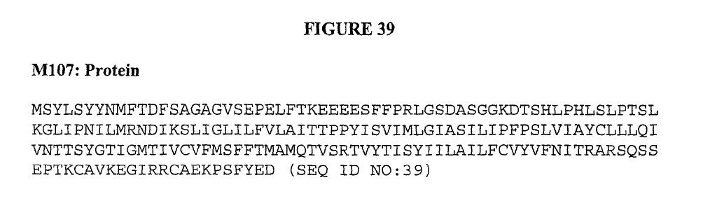

The M107 protein (SEQ ID NO:39) (FIG. 39) is predicted to contain four transmembrane regions and demonstrates high homology to multi-transmembrane receptors, particularly the 7TM receptor of C. elegans. Since, as noted above, such 7TM receptors are found among numerous chemokine receptors, including CXCR1, CCR2b, CXCR4, CCR4 and CCR5, it is herein hypothesized that the expression of M107 among infected cells leads to the disruption of chemokine receptor function. Therefore, the present invention provides the M107 polypeptide as a general immunomodulator of chemokine receptor function.

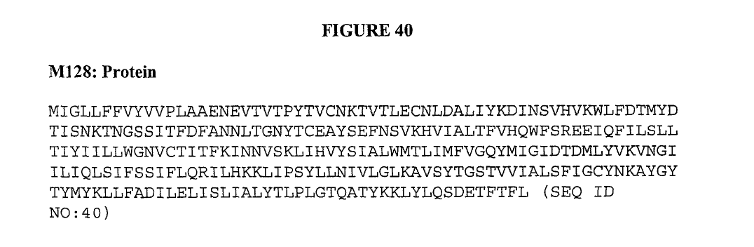

Sequence analysis of the M128 polypeptide (SEQ ID NO:40) (FIG. 40) demonstrates at least five transmembrane regions with significant homology to CD47 (also known as the integrin-associated protein; IAP). Previous research has demonstrated that CD47 provides an adhesion-dependent costimulation pathway to T cells that is independent of CD28 molecules (Walclavicek et al, J. Immunol, 159(11):5345-5354, 1997). Therefore, without wishing to be bound by any particular theory, we propose the M128 disrupts adhesion mediated costimulation of T cells during myxoma infection and prevents CD47 function. In preferred embodiments of the present invention, M128 protein is provided as an immunosuppressant. More specifically, the M128 protein is provided as a T cell specific immunosuppressor.

Leporipox Virus Protein Expression

In general, leporipox virus proteins according to the invention, may be produced by transformation of a suitable host cell with all or part of a leporipox virus protein-encoding cDNA fragment (e.g., the cDNAs described above) in a suitable expression vehicle.

Those skilled in the field of molecular biology will understand that any of a wide variety of expression systems may be used to provide the recombinant protein. The precise host cell used is not critical to the invention. The leporipox virus protein may be produced in a prokaryotic host (e.g., E. coli) or in a eukaryotic host (e.g., Saccharomyces cerevisiae, insect cells, e.g., Sf21cells, or mammalian cells, e.g., COS 1, NIH 3T3, or HeLa cells). Such cells are available from a wide range of sources (e.g., the American Type Culture Collection, Rockland, Md.; also, see, e.g., Ausubel et al., Current Protocols in Molecular Biology, John Wiley & Sons, New York, 1994). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual, P. H. Pouwels et al, 1985, Supp. 1987).

One preferred expression system is the baculovirus system (using, for example, the vector pBacPAK9) available from Clontech (Pal Alto, Calif.). If desired, this system may be used in conjunction with other protein expression techniques, for example, the myc tag approach described by Evan et al. (Mol. Cell Biol. 5:3610-3616, 1985).

Alternatively, a leporipox virus protein is produced by a stably-transfected mammalian cell line. A number of vectors suitable for stable transfection of mammalian cells are available to the public, e.g., see Pouwels et al (supra); methods for constructing such cell lines are also publicly available, e.g., in Ausubel et al. (supra). In one example, cDNA encoding the leporipox virus protein is cloned into an expression vector which includes the dihydrofolate reductase (DHFR) gene. Integration of the plasmid and, therefore, the leporipox virus protein-encoding gene into the host cell chromosome is selected for by inclusion of 0.01-300 μM methotrexate in the cell culture medium (as described in Ausubel et al., supra). This dominant selection can be accomplished in most cell types. Recombinant protein expression can be increased by DHFR-mediated amplification of the transfected gene. Methods for selecting cell lines bearing gene amplifications are described in Ausubel et al. (supra); such methods generally involve extended culture in medium containing gradually increasing levels of methotrexate. DHFR-containing expression vectors commonly used for this purpose include pCVSEII-DHFR and pAdD26SV(A) (described in Ausubel et al., supra). Any of the host cells described above or, preferably, a DHFR-deficient CHO cell line (e.g., CHO DHFR⁻ cells, ATCC Accession No. CRL 9096) are among the host cells preferred for DHFR selection of a stably-transfected cell line or DHFR-mediated gene amplification.

Once the recombinant leporipox virus protein is expressed, it is isolated, e.g., using affinity chromatography. In one example, an anti-leporipox virus protein antibody (e.g., produced as described herein) may be attached to a column and used to isolate the leporipox virus protein. Lysis and fractionation of leporipox virus protein-harboring cells prior to affinity chromatography may be performed by standard methods (see, e.g., Ausubel et al., supra). In another example, leporipox virus proteins may be purified or substantially purified from a mixture of compounds such as an extract or supernatant obtained from cells (Ausubel et al., supra). Standard purification techniques can be used to progressively eliminate undesirable compounds from the mixture until a single compound or minimal number of effective compounds has been isolated.

Once isolated, the recombinant protein can, if desired, be further purified, e.g., by high performance liquid chromatography (see, e.g., Fisher, Laboratory Techniques In Biochemistry And Molecular Biology, eds., Work and Burdon, Elsevier, 1980).

Polypeptides of the invention, particularly short leporipox virus protein fragments, can also be produced by chemical synthesis (e.g., by the methods described in Solid Phase Peptide Synthesis, 2nd ed., 1984 The Pierce Chemical Co., Rockford, Ill.).

These general techniques of polypeptide expression and purification can also be used to produce and isolate useful leporipox virus protein fragments or analogs (described herein).

In certain preferred embodiments, the leporipox protein might have attached any one of a variety of tags. Tags can be amino acid tags or chemical tags and can be added for the purpose of purification (for example a 6-histidine tag for purification over a nickel column). In other preferred embodiments, various labels can be used as means for detecting binding of a leporipox protein to another protein, for example to a chemokine or a chemokine receptor. Alternatively, leporipox DNA or RNA may be labeled for detection, for example in a hybridization assay. Leporipox virus nucleic acids or proteins, or derivatives thereof, may be directly or indirectly labeled, for example, with a radioscope, a fluorescent compound, a bioluminescent compound, a chemiluminescent compound, a metal chelator or an enzyme. Those of ordinary skill in the art will know of other suitable labels or will be able to ascertain such, using routine experimentation. In yet another preferred embodiment of the invention, the proteins disclosed herein, or derivatives thereof, are linked to toxins. Proteins linked to toxins can be used, for example to target toxic drugs to malignant tumors if the protein has the ability localize to the tumor.

Anti-leporipox Virus Protein Antibodies

To generate leporipox virus protein-specific antibodies, a leporipox virus protein coding sequence (i.e., mVOX-2) may be expressed, for example, as a C-terminal fusion with glutathione S-transferase (GST) (Smith et al., Gene 67:31-40, 1988). The fusion protein may then be purified on glutathione-Sepharose beads, eluted with glutathione cleaved with thrombin (at the engineered cleavage site), and purified to the degree necessary for immunization of rabbits. Primary immunizations are carried out with Freud's complete adjuvant and subsequent immunizations with Freud's incomplete adjuvant. Antibody titres are monitored by Western blot and immunoprecipitation analyzes using the thrombin-cleaved leporipox virus protein fragment of the GST-leporipox virus fusion protein. Immune sera are affinity purified using CNBr-Sepharose-coupled leporipox virus protein. Antiserum specificity is determined using a panel of unrelated GST proteins (including GSTp53, Rb, HPV-16 E6, and E6-AP) and GST-trypsin (which was generated by PCR using known sequences).

As an alternate or adjunct immunogen to GST fusion proteins, peptides corresponding to relatively unique hydrophilic leporipox virus proteins may be generated and coupled to keyhole limpet hemocyanin (KLH) through an introduced C-terminal lysine. Antiserum to each of these peptides is similarly affinity purified on peptides conjugated to BSA, and specificity tested in ELISA and Western blots using peptide conjugates, and by Western blot and immunoprecipitation using leporipox virus protein expressed as a GST fusion protein.

Alternatively, monoclonal antibodies may be prepared using the leporipox virus proteins described above and standard hybridoma technology (see, e.g., Kohler et al., Nature, 256:495, 1975; Kohler et al., Eur. J Immunol. 6:511, 1976; Kohler et al., Eur. J. Immunol. 6:292, 1976; Hammerling et al., In Monoclonal Antibodies and T Cell Hybridomas, Elsevier, NY, 1981; Ausubel et al., supra). Once produced, monoclonal antibodies are also tested for specific leporipox virus protein recognition by Western blot or immunoprecipitation analysis (by the methods described in Ausubel et al., supra). Antibodies which specifically recognize leporipox virus proteins are considered to be useful in the invention; such antibodies may be used, e.g., in an immunoassay to monitor the level of leporipox virus proteins produced by a mammal (for example, to determine the amount or location of a leporipox virus protein).

Preferably, antibodies of the invention are not only produced using the whole leporipox virus polypeptide, but using fragments of the leporipox virus polypeptide which lie outside highly conserved regions and appear likely to be antigenic, by criteria such as high frequency of charged residues may also be used. In one specific example, such fragments are generated by standard techniques of PCR and cloned into the pGEX expression vector (Ausubel et al., supra). Fusion proteins are expressed in E. coli and purified using a glutathione agarose affinity matrix as described in Ausubel et al. (supra). To attempt to minimize the potential problems of low affinity or specificity of antisera, two or three such fusions are generated for each protein, and each fusion is injected into at least two rabbits. Antisera are raised by injections in a series, preferably including at least three booster injections.

Identification of Molecules that Modulate Leporipox Virus Biological Activity or Whose Biological Activity is Modulated by Leporipox Virus

Isolation of the leporipox virus cDNA (as described herein) also facilitates the identification of molecules that increase or decrease a leporipox virus polypeptide biological activity. Similarly, molecules whose activity is modulated by a leporipox virus polypeptide biological activity can be identified. According to one approach, candidate molecules are added at varying concentrations to the culture medium of cells expressing leporipox virus mRNA. Leporipox virus polypeptide biological activity is then measured using standard techniques. The measurement of biological activity can include the measurement of leporipox virus polypeptide protein and nucleic acid molecule levels, or the effect of leporipox virus polypeptide on immunomodulation.

If desired, the effect of candidate modulators on expression can, in the alternative, be measured at the level of leporipox virus protein production using the same general approach and standard immunological detection techniques, such as western blotting or immunoprecipitation with a leporipox virus polypeptide-specific antibody (see below).

Candidate modulators can be purified (or substantially purified) molecules or can be one component of a mixture of compounds (e.g., an extract or supernatant obtained from cells; Ausubel et al., supra). In a mixed compound assay, leporipox virus polypeptide expression is tested against progressively smaller subsets of the candidate compound pool (e.g., produced by standard purification techniques, e.g., HPLC or FPLC) until a single compound or minimal compound mixture is demonstrated to modulate leporipox virus polypeptide expression.

Alternatively, or in addition, candidate compounds can be screened for those that modulate leporipox virus polypeptide activity. In this approach, the level of immunomodulation in the presence of a candidate compound is compared to the level of immunomodulation in its absence, under equivalent conditions. Again, such a screen can begin with a pool of candidate compounds, from which one or more useful modulator compounds is isolated in a step-wise fashion.

The screening assays described above can be carried out in a variety of ways that are well known to those skilled in this art. These include using leporipox virus polypeptide variants or by using fragments of a leporipox virus polypeptide.

A test compound that can be screened in the methods described above can be a chemical, be it naturally-occurring or artificially-derived. Such compounds can include, for example, polypeptides, synthesized organic molecules, naturally occurring organic molecules, nucleic acid molecules, and components thereof. Candidate leporipox virus polypeptide modulators include peptide as well as non-peptide molecules (e.g., peptide or non-peptide molecules found, e.g., in a cell extract, mammalian serum, or growth medium in which mammalian cells have been cultured).

In general, novel drugs for prevention or treatment of immunomodulatory diseases are identified from large libraries of both natural products, synthetic (or semi-synthetic) extracts, and chemical libraries using methods that are well known in the art. Those skilled in the field of drug discovery and development will understand that the precise source of test extracts or compounds is not critical to the screening methods of the invention. Accordingly, virtually any number of chemical extracts or compounds can be screened using these methods. Examples of such extracts or compounds include, but are not limited to, plant-, fungal-, prokaryotic-, or animal-based extracts, fermentation broths, and synthetic compounds, as well as modifications of existing compounds.

Numerous methods are also available for generating random or directed synthesis (e.g., semi-synthesis or total synthesis) of any number of chemical compounds, including, but not limited to, saccharide-, lipid-, peptide-, and nucleic acid molecule-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound can be readily modified using standard chemical, physical, or biochemical methods.

In addition, those skilled in the art of drug discovery and development readily understand-that methods for dereplication (e.g., taxonomic dereplication, biological dereplication, and chemical dereplication, or any combination thereof) or the elimination of replicates or repeats of materials already known for their therapeutic activities for immunological disorders can be employed whenever possible.

When a crude extract is found to regulate immunomodulation, further fractionation of the positive lead extract can be carried out to isolate chemical constituents responsible for the observed effect. Thus, the goal of the extraction, fractionation, and purification process is the careful characterization and identification of a chemical entity within the crude extract having a desired activity. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value can be subsequently analyzed using, for example, any of the animal models described herein.

Testing and Administration of Immunomodulatory Molecules

Leporipox immunomodulatory polypeptides of the present invention may be screened for immunomodulatory activity. For example, chemotaxis activity in the presence of a candidate compound is compared to chemotaxis activity in its absence, under equivalent conditions. Such a screen may begin with a pool of candidate compounds, from which one or more useful immunomodulator compounds are isolated in a step-wise fashion. Chemotaxis (of eosinophil or other leukocyte) activity may be measured by any standard assay, for example, those described herein.

Candidate leporipox virus encoded modulators include peptide as well as non-peptide molecules (e.g., peptide or non-peptide molecules found, e.g., in a cell extract, mammalian serum, or growth medium on which mammalian cells have been cultured). Particularly useful are modulators of leporipox virus protein expression.

A molecule which promotes an increase in leporipox virus protein expression or a decrease in leukocyte chemotaxis activity is considered particularly useful in the invention; such a molecule may be used, for example, as a therapeutic to decrease the immunoreactivity in a individual. In addition, leporipox virus proteins identified in the present invention (or modifications and derivatives thereof) that exhibit these activities are also particularly desirable. For example, these polypeptides may be used to treat an individual that has rheumatoid arthritis. Other human diseases that may be treated using a molecule that act as an immunosuppressant or otherwise reduces the immune function include, acute inflammation, allergic reactions, asthmatic reactions, inflammatory bowel diseases (i.e., Crohn's Disease and ulcerative colitis), transplant rejection, and restenosis.

A molecule which enhances or induces apoptosis may alternatively be used in the treatment of tumors.

Immunomodulators of this invention and other polypeptides found to be effective at the level of leporipox virus protein expression or activity may be confirmed as useful in animal models. The animal models that may be used in the present invention will test drug candidates for efficacy in treating autoimmune and inflammatory diseases. Target therapeutic areas include acute inflammation, rheumatoid arthritis, transplant rejection, asthma, inflammatory bowel disease, uveitis, restenosis, multiple sclerosis, psoriasis, wound healing, lupus erythematosus and any other autoimmune or inflammatory disorder that can be recognized by one of ordinary skill in the art.

For example, other diseases related to inflammation that may be treated by methods the present invention include, for example, allergic rhinitis, atopic dermatitis and food allergies. Examples of other autoimmune disorders, where the immune system attacks the host's own tissues, include, but are not limited to type 1 insulin-dependent diabetes mellitus, deramatitis, meningitis, thrombotic thrombocytopenic purpura, Sjogren's syndrome, encephalitis, leukocyte adhesion deficiency, rheumatic fever, Reiter's syndrome, psoriatic arthritic, progressive systemic sclerosis, primary biliary cirrhosis, pemphigus, pemphigoid, necrotizing vasculitis, mayasthenia gravis, lupus erythmatosus, polymyositis, sarcoidosis, granulomatorsis, vasculitis, pernicious anemia, CNS inflammatory disorder antigen-antibody complex mediated diseases, autoimmune hemolytic anemia, Hashimoto's thyroiditis, Graves disease, habitual spontaneous abortions, Reynard's syndrome, glomerulonephritis, dermatomyositis, chromic active hepatitis, celiac disease, autoimmune complications of AIDS, atrophic gastritis, ankylosing spondylitis and Addison's disease.

Other diseases related to non-malignant or immunological-related cell-proliferative diseases that may be treated by methods the present invention include for example psoriasis, penphigus vularis, Behcet's syndrome, acute respiratory distress syndrome (ARDS), ischemic heart disease, atherosclerosis, post-dialysis syndrome, leukemia, acquired immune deficiency syndrome septic shock and other type of acute inflammation, and lipid histiocytosis.

If successful, the identified leporipox virus polypeptide may be used as anti-inflammatory or anti-cancer therapeutics (e.g., a mouse tumor model).

Animal models for testing the immunomodulatory effects of candidate compounds are well known in the art. Therefore, the present invention merely refers to a selection of animal models that can be used to test the candidate compounds o the invention and omits what is well known in the art. Animal models proposed for use in the present invention to test candidate compounds for their efficacy in treating autoimmune and inflammatory disorders include, but are not limited to:

Acute Inflammation:

Animal models of acute inflammation are targeted for initial, fast drug efficacy screening and for their potential predicative value of outcomes in chronic inflammatory diseases. The following animal models may be used to test the candidate compounds of the present invention for their efficacy in treating acute inflamation: 1) carrageenin induced inflammation 2) turpentine induced inflammation 3) transgenic HLAB-27 inflammatory and 4) ear-scratch model of inflammation.

Rheumatoid Arthritis; Rat, Mouse and Rabbit

Efficacy in Rheumatoid Arthritis evaluated in 1) various antigen induced arthritis models in rabbit, rat and mouse; and 2) in transgenic rheumatological models.

The molecular and cellular mechanisms of action of the candidate compounds will be evaluated by testing their efficacy in influencing key intracellular mechanisms that regulate degradative processes involved in joint disease. Important molecular and cellular mechanisms that will receive particular focus include signaling events regulating disease processes such as increased angiogenesis, synovial hyperplasia and matrix metalloprotease expression. These processes are thought to be involved in cartilage degradation in arthritic diseases.

1. Collagen Induced Arthritis; Rat, Mouse and Rabbit

Autoimmune-mediated polyarthritis can be induced in certain strains of rodents (rat, mouse and rabbit) and non-human primates by immunizing them with native type II collagen. The collagen induced arthritis model is widely used and well characterized. Collagen induced arthritis is mediated by susceptibility to autoantibodies which bind to a particular region of type II collagen. The mechanism of induction is linked to MHC-class II molecules but also depends on the species of type II collagen used for immunization.

2. Ovalbumin Induced Arthritis; Rabbit

Candidate compounds are tested for efficacy in decreasing signs and symptoms of ovalbumin arthritis. Polyarthritis is induced in rabbits by immunizing them with Ovalbumin.

3. Adjuvant Induced Arthritis; Rat, Mouse and Rabbit

Candidate compounds are tested for the efficacy in decreasing signs and symptoms of adjuvant induced arthritis. Polyarthritis is induced in certain strains of rodents by immunizing them with Freud's Adjuvant.

4. Streptococcal Cell Wall-induced Arthritis; Rat

Candidate compounds are tested for efficacy in decreasing signs and symptoms of streptococcal cell wall-induced arthritis. Chronic, erosive polyarthritis is induced by intraperitoneal-injection of aqueous suspension of cell wall fragments, isolated from group A streptococci.

Transplant Rejection (Acute and Chronic)

Efficacy in transplant rejection is evaluated in various models of graft vascular disease (GVD). GVD is the most common cause of late graft failure in solid organ transplantation. GVD or graft atherosclerosis is characterized by plaque formation and fibrosis in small vessels. The development of graft vascular disease has been associate with acute allograft rejection, ischemia-reperfusion injury and bacterial or viral infections. The common pathway of these postoperative insults results in perivascular inflammation which triggers migration of mesenchymal cells into the vessel wall eventually resulting in occlusion or partial occlusion of the vessel lumen.

1. Aortic Allograft Model; Rat, Rabbit, Monkey

Candidate compounds are tested for efficacy in reducing graft atherosclerosis and transplant rejection in a model of vascular injury after transplantation of aortic segments performed in certain strains of MHC mismatched rats, and rabbits.

2. Tracheal Allograft Model; Rat, Rabbit, Monkey

Candidate compounds are tested for efficacy in reducing graft atherosclerosis and transplant rejection in a model of vascular injury after transplantation of tracheal segments performed in certain strains of MHC mismatched rats, rabbits, and monkeys.

3. Heterotopic Heart Transplant; Mouse, Rat, Monkey

A heterotropic heart transplantation is performed in MHC mismatched rats. In this model, rodents are treated with cyclosporine A for only the first 7 days after transplantation, are allowed to develop graft vascular disease, and are then analyzed after sacrifice at postoperative day 90.

4. Orthotopic Kidney Transplant; Mouse, Rat, Monkey

An orthotopic kidney transplantation is performed in MHC mismatched rats. In this model, animals receiving subtherapeutic doses of cyclosporine A for the first 10 days after transplantation, are allowed to exhibit features of chronic renal allograft rejection in 70% of cases, and are then analyzed after sacrifice at postoperative day.

5. Orthotopic Lung Transplant; Rat, Monkey

Candidate compounds are tested for effectiveness in delaying or reducing signs and symptoms of organ rejection after lung whole organ transplantation in rats and monkeys

6. Reperfusion Injury; Rat

The immediate postoperative course in clinical lung transplantation is often severely impaired by delayed graft function as a result of ischemia and reperfusion injury. Preventive efficacy of drug candidates in ischemia-reperfusion injury is evaluated using a model of acute, in vivo double lung transplantation in the rat (Hausen et al., Ann Thorac Surg 61:1714-9,1996 incorporated herein by reference).

Restenosis

Candidate compounds will be tested for efficacy in reducing atherosclerotic plaque deposition in a model of coronary restenosis after balloon angioplasty. Atherosclerotic plaque formation is critically involved in vascular occlusion and has been linked to excessive inflammatory and thrombotic response to arterial injury.

Asthma; Rodent

The effectiveness of candidate compounds in reduction of signs and symptoms of asthma is evaluated in rodent models of antigen induced experimental airways inflammation. The models include:

1. Ovalbumin Induced Experimental Airways Inflammation; Rodent

Candidate compounds are tested for efficacy in reducing inflammatory cell components in the bronchoalveolar lavage of the lungs after aerosol challenge in ovalbumin sensitized rodent models of experimental airways inflammation.

2. Ovalbumin Induced Allergic Sensitization in Presence of GM-CSF Transgene Expression; Mice

Candidate compounds are tested for efficacy in reducing inflammatory cell components in the bronchoalveolar lavage of the lungs of mice after ovalbumin aerosol challenge in the context of local expression of GM-CSF (Staempfli et al., J. Clin. Invest., Vol. 102:9, 1704-1714).

Inflammatory Bowel Disease (IBD); Mice and Rats

Drug candidates are evaluated for their potential therapeutic efficacy in ulcerative colitis or Crohn's disease utilizing various models of antigen induced and genetically mediated spontaneous chronic intestinal inflammation in mice and rats. Examples include:

1. Dextran Sulfate Sodium Induced IBD; Mice

Chronic, irreversible clinical symptoms of IBD are induced by treating mice with an oral administration of dextran sulfate sodium.

2. Gene Deletion and Transgenic Models for IBD; Rodent

Compound efficacy will be tested in transgenic rodent lines which develop symptoms closely resembling the human elements of inflammatory bowel disease. Models include, the targeted deletion of the genes encoding IL-2, IL-10, TGF beta, T-cell receptor alpha/beta, keratin 8, Gi2 alpha. In addition, animals expressing transgenes for the human WA-B27 and HLA-B27 as well as a dominant negative construct which functionally blocks N-cadherin will be tested.

Uveitis

Drug candidates will be evaluated for efficacy in various animal models of uveitis. Key models include both, experimental autoimmune uveitis and adoptively transferred experimental autoimmune uveitis.

1. Experimental Autoimmune Uveitis (EAU)

EAU is a T-cell mediated inflammatory eye disease that can be induced in several mammalian species by immunization with ocular-specific antigens (Gery et al., Invest. Opthalmol. Vis. Sci., 27: 1296-1300, 1986,. Sanui et al., J. Exp. Med., 169:1947-1989, incorporated herein by reference). This experimental disease is considered a model for a family of inflammatory eye diseases in humans and has been used to examine numerous modalities before their human testing.

2. Adoptively Transferred Experimental Autoimmune Uveitis

Adoptively transferred EAU is induced through injection of lymphocytes presensitized against the retinal antigen are injected into naive syngenic recipients (McAllister et al., J. Immunol., 138:1416-1420, 1987 incorporated herein by reference).

Once identified, a leporipox virus immunomodulator or anticarcinogen may be administered with a pharmaceutically-acceptable diluent, carrier, or excipient, at a pharmaceutically effective dose. Conventional pharmaceutical practice may be employed to provide suitable formulations or compositions to administer leporipox virus proteins to patients suffering from presymptomatic carcinoma. Any appropriate route of administration may be employed, for example, parenteral, intravenous, subcutaneous, intramuscular, intracranial, intraorbital, ophthalmic, intraventricular, intracapsular, intraspinal, intracistemal, intraperitoneal, intranasal, aerosol, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found in, for example, “Remington's Pharmaceutical Sciences.” Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for NES1 modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

If desired, treatment with a NES 1 modulatory compound may be combined with more traditional cancer therapies such as surgery, radiation, or chemotherapy.

Detection of Specific Conditions

Leporipox virus polypeptides and nucleic acid sequences find diagnostic use in the detection or monitoring of inflammatory, autoimmune and other conditions. For example, because leporipox virus proteins are involved in leukocyte chemotaxis and because a decrease in the number of leukocytes correlates with immunosuppression, an alteration in the level of particular leporipox virus protein production provides an indication of the prognosis of the condition. Levels of leporipox virus protein expression may be assayed by any standard technique. For example, its expression in a biological sample (e.g., a biopsy) may be monitored by standard Northern blot analysis or may be aided by PCR (see, e.g., Ausubel et al., supra; PCR Technology: Principles and Applications for DNA Amplification, ed., H. A. Ehrlich, Stockton Press, NY; and Yap and McGee, Nucl. Acids. Res. 19:4294, 1991).

In yet another approach, immunoassays are used to detect or monitor leporipox virus protein in a biological sample. Leporipox virus protein-specific polyclonal or monoclonal antibodies (produced as described above) may be used in any standard immunoassay format (e.g., ELISA, Western blot, or RIA assay) to measure leporipox virus polypeptide levels; again comparison is to wild-type leporipox virus protein levels. A change in leporipox virus protein production may be indicative of a particular prognosis. Examples of immunoassays are described, e.g., in Ausubel et al., supra. Immunohistochemical techniques may also be utilized for leporipox virus protein detection. For example, a tissue sample may be obtained from a patient, and a section stained for the presence of leporipox virus protein using an anti-leporipox virus protein antibody and any standard detection system (e.g., one which includes a secondary antibody conjugated to horseradish peroxidase). General guidance regarding such techniques can be found in, e.g., Bancroft and Stevens (Theory and Practice of Histological Techniques, Churchill Livingstone, 1982) and Ausubel et al. (supra).

Leporipox Virus Gene Therapy

Because expression of leporipox virus protein may correlate with autoimmune, inflammatory or tumor prognosis, the leporipox virus gene also finds use in immunomodulatory or anti-cancer gene therapy. For example, to enhance leukocyte infiltration of a tumor, a functional leporipox virus gene may be introduced into cells at the site of a tumor. In addition, leporipox virus polypeptides that are shown to reverse autoimmune reactions may also be used in gene therapy. Alternatively, leporipox virus polypeptides which alterations which block inflammatory may be administered via gene therapy for the treatment of eosinophil mediated inflammatory conditions.

Retroviral vectors, adenoviral vectors, adeno-associated viral vectors, or other viral vectors with the appropriate tropism for leporipox virus protein-expressing cells may be used as a gene transfer delivery system for a therapeutic leporipox virus gene construct. Numerous vectors useful for this purpose are generally known (Miller, Human Gene Therapy 15-14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cometta et al., Nucleic Acid Research and Molecular Biology 36:311-322, 1987; Anderson, Science 226:401-409, 1984; Moen, Blood Cells 17:407-416, 1991; and Miller and Rosman, Biotechniques 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches may also be employed for the introduction of therapeutic DNA into cells. For example, leporipox virus gene may be introduced into a tumor cell by the techniques of lipofection (Felgner et al., Proc. Natl. Acad. Sci. USA 84:7413, 1987; Ono et al., Neuroscience Lett 117:259, 1990; Brigham et al., Am. J Med. Sci. 298:278, 1989; Staubinger and Papahadjopoulos, Meth. Enz. 101:512, 1983); asialorosonucoid-polylysine conjugation (Wu and Wu, J. Biol. Chem. 263:14621, 1988; Wu et al., J. Biol. Chem. 264:16985, 1989); or, less preferably, microinjection under surgical conditions (Wolff et al., Science 247:1465, 1990).

For any of the above approaches, the therapeutic leporipox virus DNA construct is preferably applied to the site of the malignancy or inflammation and cytotoxic damage (for example, by injection), but may also be applied to tissue in the vicinity of the malignancy or inflammation and cytotoxic damage or even to a blood vessel supplying these areas.

In the gene therapy constructs, leporipox virus cDNA expression is directed from any suitable promoter (e.g., the human cytomegalovirus, simian virus 40, or metallothionein promoters), and its production is regulated by any desired mammalian regulatory element. For example, if desired, enhancers known to direct preferential gene expression in endothelial or epithelial cells may be used to direct leporipox virus protein expression. Such enhancers include, without limitation, the lung specific promotors (e.g. surfactant), and gut specific regulatory sequences.

Alternatively, if a leporipox virus genomic clone is utilized as a therapeutic construct (for example, following its isolation by hybridization with the leporipox virus cDNA described above), leporipox virus protein expression is regulated by its cognate regulatory sequences or, if desired, by regulatory sequences derived from a heterologous source, e.g., any of the promoters or regulatory elements described above.

Less preferably, leporipox virus gene therapy is accomplished by direct administration of the leporipox virus mRNA to a tumor. This mRNA may be produced and isolated by any standard technique, but is most readily produced by in vitro transcription using a leporipox virus cDNA under the control of a high efficiency promoter (e.g., the T7 promoter). Administration of leporipox virus mRNA to malignant cells is carried out by any of the methods for direct nucleic acid administration described above.

Ideally, the production of leporipox virus protein by any gene therapeutic approach described above results in a cellular level of leporipox virus protein that is at least equivalent to the normal, cellular level of NES1 in an unaffected individual. Treatment by any NES1-mediated gene therapy approach may be combined with more traditional cancer therapies (e.g., surgery, radiation, or chemotherapy for treatment of tumors).

Another therapeutic approach included within the invention involves direct administration of recombinant leporipox virus protein, either to the site of a malignancy (for example, by injection) or systemically by any conventional recombinant protein administration technique for treatment of an autoimmune or inflammatory disorder. The actual dosage of leporipox virus protein depends on a number of factors, including the size and health of the individual patient, but, generally, between 0.1 mg and 100 mg inclusive are administered per day to an adult in any pharmaceutically-acceptable formulation.

The above approaches may also be used to inhibit the activity of the candidate compound by substituting an altered leporipox virus polypeptide having leporipox virus protein blocking activity (e.g., have a deletion or insertion at the amino terminus) for the leporipox virus polypeptide described above.

Transgenic Animals

Transgenic animals may be made using standard techniques. For example, a leporipox virus gene may be provided using endogenous control sequences or using constitutive, tissue-specific, or inducible regulatory sequences. Transgenic animals lacking functional leporipox virus polypeptide may also be made using standard techniques. This may be done by engineering knock-out mutations in the leporipox virus gene using DNA sequences provided herein.

Leporipox Virus Gene Sequences

In general, the present invention relates to leporipox virus nucleic acid. In some preferred embodiments the nucleic acid is genomic DNA. In other preferred embodiments the nucleic acid is cDNA. In yet other preferred embodiments, the nucleic acid is mRNA. In certain preferred aspects of the invention the leporipox virus nucleic acid encodes an immunomodulatory polypeptide. Preferably the immunomodulatory polypeptide is of the myxoma species or alternatively the Shope fibroma species of leporipox viruses. The immunomodulatory polypeptide may be an immunosuppressor or an immunostimulator. Preferably the invention relates to a polypeptide that is substantially identical (at least 80% identity) with the polypeptides disclosed herein. For example, the polypeptide may be any one, or a combination of a cytokine, an anti-inflammatory, an immunoreceptor, a multi-transmembrane receptor protein or a secreted protein.

Myxoma Virus Knockouts

Poxviruses are among the largest eukaryotic DNA viruses and have the unusual capacity to replicate autonomously in the cytoplasm of infected cells. Many poxvirus proteins have been defined as virulence factors on the basis that, when present, they confer increased pathogenicity and improve viral replication within immunocompetent hosts. When genes that encode these virulence factor proteins are deleted, the resulting virus strain generally exhibits an attenuated or altered disease phenotype (Turner, Curr. Top. Microbiol. Immunol., 163:125-151, 1990; Buller Microbiol. Rev., 55:80-122, 1991; Smith, J Gen. Virol., 74:1725-1740; McFadden, Austin (Tex.): R. G. Landes Company, 1995, incorporated herein by reference). Such “knockout” myxoma viruses may assist in the elucidation of the immunomodulatory or other role of viral proteins.

Using standard virological assays (Nash et al., (1999) Immunological Review, 57:731 to end), one of ordinary skill in the art may establish the contribution of each gene to myxoma virus infection and the general biochemical and physiological progression of myxomatosis. Knockout myxoma viruses lacking a particular gene may be generated using standard molecular biological techniques (see Sambrook et al., Molecular Cloning: A Laboratory Manual, 2^{nd} Ed., Cold Spring Harbor Press, Cold Spring Harbor, N.Y., 1989; Innis et al., PCR Protocols: A Guide to Methods and Applications, Academic Press, San Diego, Calif., 1990,; Erlich et al., PCR Technology: Principles and Applications for DNA Amplification, Stockton Press, New York, N.Y., 1989 each of which is incorporated herein by reference). The virulence of these knockout viruses may be assessed using standard infectivity assays well known in the art (see Nash et al., (1999) Immunological Review, 57:731 to end). For example, as mentioned above, the OX-2 homolog M141 (mVox-2) (SEQ ID NO: 9) might be useful in elucidating the mechanism used by myxoma virus and other poxviurses to disrupt T cell stimulation. Such a discovery may lead to a method of modulating T cell stimulation in humans.

### Examples

Cloning Additional Leporipox Virus Genes.

Based on the amino acid sequences provided by the present invention degenerate oligonucleotide primers containing restriction sites will be synthesized. First strand cDNA will be synthesized from RNA prepared from a tissue of interest and PCR will be performed with an initial five cycles, for example, at 37° for 60s, followed by 25 cycles at 50° for 60s (denaturation at 95° for 30 s and extension at 72° for 90 s) in order to amplify a leporipox virus cDNA fragment that can be subsequently subcloned into Bluescript II KS (Stratagene). Construction of a cDNA library using poly A RNA isolated from the selected tissue will be performed using Stratagene ZAP Express Vector according to the directions of the manufacturer. Potential clones will be subsequently amplified and an aliquot of this cDNA library containing phage will be screened with the leporipox virus cDNA that has been 32P-labeled with Klenow enzyme. Isolated phagemids will be subsequently subjected to automated sequencing on both strands using Applied Biosystems Instrumentation (model 373a) and the dye-terminator protocol. Sequence analysis will be performed using software developed by the University of Wisconsin genetics computer group (Altschul, et al. J. Mol. Biol. 215:403-410, 1990).

DNA and RNA Analysis.

RNA will be isolated by CsCl centrifugation in guanidine isothiocyanate (Chirgwin, et al. (1979) Biochemistry. 18:5294-9). Biologically active ribonucleic acid will be isolated from sources enriched in ribonuclease. DNA will be isolated from these gradients as well. In some cases, RNA will be isolated using RNAzol (Biotecx Lab, Inc.) according to the directions of the manufacturer. Poly A RNA will be enriched by elution through an oligo dT column (Pharmacia). For example, 10 mcg of total RNA, 2 mcg of poly A RNA, or 10 mcg of restriction endonuclease cut DNA will be electrophoresed in agarose, and transferred to Gene Screen (NEN Dupont) membranes. Membranes will be hybridized with ³²P labeled full length cDNA or a fragment encoding the translated protein. High stringency hybridization will be performed, for example, in 50% formamide, 10% dextran sulfate, 5× SSC, 1× Denhardt's solution (0.0002% (w/v) polyvinylpyrrolidone, 0.0002% (w/v) BSA, 0.0002% (w/v) Ficoll 400), 1% (w/v) SDS, 100 mcg/ml denatured herring sperm DNA, and 20 mM Tris at 42o C. and blots will be washed with 0.2× SSC, 0.5% SDS at 65o C. Low stringency hybridization will be performed, for example, in 0.6M NaCl, 80 mM TrisCl, 4mM EDTA, 0.1% (w/v) sodium pyrophosphate, 0.1% (w/v) SDS, 10× Denhardts, 100 mcg/ml denatured herring sperm DNA at 50o C. and washed with 1× SSC, 0.05% SDS at 50o C. Quantitation of the intensity of band hybridization will be determined using a Phosphor-Imager (Molecular Dynamics).

Leporipox Virus Gene Analysis.

A cDNA probe from the coding region of the Leporipox virus cDNA will be ³²P-labeled with Klenow enzyme and used to screen approximately 1×106 plaques from a mammalian genomic library (e.g., a variety of libraries are available from Stratagene, La Jolla, Calif.) under conditions of low stringency (hybridization in 0.6 M NaCl, 80 mM TrisCl, 4 mM EDTA, 0.1% sodium pyrophosphate, 0.1% SDS, 10× DenhardtOs solution (0.002% polyvinylpyrrolidone, 0.002% BSA, 0.002% Ficoll 400), 100 mcg/ml denatured herring sperm DNA at 50° C. and blots washed with 1× SSC, 0.05% SDS at 50o C.). Plaques that hybridize strongly will be purified. The genomic DNA will be liberated from the phage DNA by restriction digestion with the appropriate enzymes and sub-cloned into pBlue-Script SK II (Stratagene). Any positively identified genomic fragment that hybridizes with the probe will be subcloned into, for example, pBlue-Script KS II, and subjected to automated sequencing on both strands using Applied Biosystems Instrumentation (model 373a) and the dye-terminator protocol. Sequence analysis will be performed using software developed by the University of Wisconsin genetics computer group Altschul, et al. (1990) J. Mol. Biol. 215, 403-410.

The leporipox virus gene homolog chromosomal localization will be determined by the analysis of any identified polymorphisms in the gene. PCR primers flanking this polymorphism will be constructed and genomic DNA will be amplified by PCR. Using these primers, a size polymorphism will be identified (For example, see Rowe, et al., Mamm. Gen. 5, 253-274, 1994).

Murine Leporipox cDNA Analysis.

The identified genomic fragment will be used to screen a mammalian cDNA expression library (Stratagene) under conditions of high stringency (hybridization in 50% formamide, 10% dextran sulfate, 5× SSC, 1× DenhardtOs solution, 1% SDS, 100 mcg/ml denatured herring sperm DNA, and 20 mM Tris at 42° C. and blots were washed with 0.2× SSC, 0.5% SDS at 65° C.). Positive plaques will be identified, purified, and phagemids will be prepared according to the instructions of the library manufacturer. The inserts will be completely sequenced on both strands by automated sequencing. Alignment analysis will be determined by the Clustal method using MegAlign software (DNASTAR Inc) (Higgins, et al. (1988) Gene 73, 237-244).

Construction and Transfection of Protein Expression Vectors.

PCR primers will be designed to amplify the coding region of the leporipox gene, or homologor derivative thereof, flanked by convenient restriction sites for subsequent sub-cloning. PCR will be performed under standard conditions using leporipox cDNA-pBlueScript as a template. The resulting PCR products will be subsequently subcloned using, for example, a TA cloning kit (Invitrogen, San Diego, Calif.) and confirmatory sequencing will be performed. Leporipox cDNA will be subcloned into the available restriction sites of pcDNA-I/Amp (Invitrogen). Approximately 4 mcg of the leporipox-pcDNA-I construct will be transfected into 100 mm plates containing ˜30% confluent COS cells using DEAE-Dextran (Lopata, et al., Nucl. Acids Res. 12, 5707-5717, 1984). In order to measure transfection efficiency, a replicate sample of COS cells will be transfected with a CMV promoter-placental alkaline phosphatase control plasmid (Fields-Berry, et al., Proc. Natl. Acad. Sci. U.S.A. 89, 693-697, 1992). RNA expression will be confirmed by Northern analysis using the leporipox cDNA as a probe. Leporipox-pcDNA-I transfected or mock transfected COS cell supernatant will be obtained after 72 hrs of culture and stored at 4° C. In another set of transfection experiments, leporipox cDNA will be similarly subcloned into the available site of MoLTR-SV40 I/PA expression vector (for example, see Luster, et al, J. Exp. Med. 178, 1057-1065, 1993). 20 mcg of linearized leporipox-MoLTR construct and 1 mcg of linearized neomycin resistance plasmid pSV7Neo will be used to transfect J558L cells by electroporation. G418 resistant cells from single wells will be analyzed for leporipox mRNA expression by Northern analysis. Cells expressing leporipox virus protein or control untransfected cells (that do not express leporipox virus protein) will be expanded in large cultures. In order to optimize the concentration of leporipox virus protein in the supernatant, the cells will be grown at high density (1×106 cells/ml) in RPMI without FCS, cultured for 72 hrs, and the conditioned medium will be concentrated 5-fold with Centricon 3000 microconcentrators (Amicon, Beverly, Mass.) before being stored.

Chemotaxis Assays.

Murine leukocytes, for example eosinophils, will be isolated. Eosinophils in particular will be isolated from IL-5 transgenic mice (Dent, et al. (1990) J. Exp. Med. 172, 1425-1431). These mice develop splenomegaly with eosinophils accounting for ˜30% of the splenocytes. Eosinophils will be purified from the spleen using immuno-magnetic separation to remove the contaminating splenocytes. Briefly, splenocytes will be labeled with anti-Thy-1 (M5/49), anti-B220 (6B2), and anti-Lyt-2 (53-6.7). Hybridoma cell lines will be obtained, for example, from American Type Culture Collection and hybridoma cell supernatants will be used as a source of antibodies. The antibody labeled cells will be treated with, anti-serum coated-magnetic beads, the anti-serum having specificity for the isotype of the primary antibody, (M450, Dynal, Great Neck, N.Y.) and eosinophils will be enriched by negative selection through a magnetic field. Macrophage cells will be isolated from the peritoneal cavity of mice that had been pre-treated (2 days prior) with intraperitoneal injection of 2.9% thioglycollate (Difco, Detroit, Calif.). Peritoneal neutrophils will be isolated from mice pre-treated with sodium casein (Luo, et al., J. Immun. 153, 4616-4624, 1994). Macrophages and neutrophils will be purified by Percoll gradients (Luo, et al., J. Immun. 153, 4616-4624, 1994. Eosinophils or macrophages will be suspended in HBSS with 0.05% BSA at 2×106 cells/ml, respectively, and 50 ml of replicate cells will be placed in the top well of a 48 well micro-chemotaxis chamber (Neuro Probe, Inc, Cabin John, Md.). A polycarbonate filter with 5-μm pores will be used to separate the cells from buffer (30 ml) alone or buffer containing recombinant COS cell supernatant and a positive (for example MCP-1 (Rollins, & Pober, Am. J Path., 138, 1315-1319, 1991)) and negative control (for example, supernatant from mock-transfected COS cells) so that a comparison can be made and the immunosuppressive (inhibition of chemotaxis) properties of the leporipox virus protein assessed. Cells will be incubated at 37o C for 60 minutes (eosinophils and neutrophils) or 90 minutes (macrophages) and the cells that migrate across the filter and adhere to the bottom side of the filter will be stained with Diff-Quick (Baxter Scientific, McGaw Park, Ill.). The number of cells per 400× field will be counted.

Statistical Analysis.

The statistical significance of differences between means will be determined by analysis of variance (ANOVA). P<0.05 will be considered significant. When ANOVA indicates a significant difference, the Newman-Keuls test will be used to determine which groups are significantly different from each other.

Analysis of Leporipox cDNA.

Using degenerate oligonucleotide primers based upon the amino acid sequence of the leporipox gene sequences disclosed herein, a cDNA will be amplified by PCR from single stranded cDNA from a selected mamamlian tissue. This PCR product will encode a peptide identical to the leporipox gene and will be used to screen an amplified cDNA library made from selected mammalian tissue. Positive plaques will be subsequently purified. Sequence analysis of the plaques will then be preformed as described herein above.

Leporipox mRNA Expression in Different Organs.

Northern blot analysis of total RNA isolated from different mammalian tissue samples may reveal detectable expression of any leporipox virus gene, homolog or derivative. Mammalian tissues including the brain, bone marrow, skin, intestines, stomach, heart, thymus, lymph node, mammary gland, skeletal muscle, tongue, spleen, liver, testes, and kidney will be analyzed. Likewise, cell lines such as macrophages isolated and cultured from the spleen, a lung epithelial cell line, and a colon adenocarcinoma cell, will be analyzed for expression of leporipox or homologous mRNA.

### Other Embodiments

In other preferred embodiments, the invention includes any protein which is substantially identical to a leporipox virus polypeptide (preferably sequences of FIGS. 21-40); such homologs include other substantially pure naturally occurring mammalian homologs of leporipox proteins as well as allelic variations; natural mutants; induced mutants; proteins encoded by DNA that hybridizes to the leporipox sequences of FIGS. 1-20 under high stringency conditions or low stringency conditions (e.g., washing at 2× SSC at 40° C. with a probe of at least 40 nucleotides); and polypeptides or proteins specifically bound by antisera directed to a leporipox polypeptide. The term also includes chimeric polypeptides that include a leporipox fragment.

The invention further includes analogs of the leporipox virus polypeptides. Analogs can differ from the naturally occurring leporipox protein by amino acid sequence differences, by post-translational modifications, or by both. Analogs of the invention will generally exhibit at least 70%, more preferably 80%, even more preferably 90%, and most preferably 95% or even 99%, identity with all or part of a naturally occurring leporipox sequence. The length of comparison sequences will be at least 8 amino acid residues, preferably at least 24 amino acid residues, and more preferably more than 35 amino acid residues. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally occurring leporipox polypeptide by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, hereby incorporated by reference; or Ausubel et al., supra, hereby incorporated by reference). Also included are cyclized peptides molecules and analogs which contain residues other than L-amino acids, e.g., D-amino acids or non-naturally occurring or synthetic amino acids, e.g., β or γ amino acids.

In addition to full-length polypeptides, the invention also includes leporipox polypeptide fragments. As used herein, the term “fragment” means at least 10 contiguous amino acids, preferably at least 30 contiguous amino acids, more preferably at least 50 contiguous amino acids, and most preferably at least 60 to 80 or more contiguous amino acids. Fragments of leporipox virus proteins can be generated by methods known to those skilled in the art or may result from normal protein processing (e.g., removal of amino acids from the nascent polypeptide that are not required for biological activity or removal of amino acids by alternative mRNA splicing or alternative protein processing events).

Preferable fragments or analogs according to the invention are those which exhibit biological activity (for example, the ability to act as an immunomodulator as described herein). Preferably, a leporipox polypeptide, fragment, or analog exhibits at least 10%, more preferably 30%, and most preferably, 70% or more of the biological activity of a full length naturally occurring leporipox virus polypeptide.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

What is claimed is:

1. A method of immunomodulation in a mammal, said method comprising: administering to said mammal a therapeutically effective amount of a leporipox virus polypeptide or fragment thereof, wherein said polypeptide has an immunomodulatory effect in said mammal.

2. The method of claim 1, wherein said leporipox virus polypeptide comprises an amino acid sequence substantially identical to SEQ ID NO: 31 or fragments and analogs thereof.

3. The method of claim 1, wherein said immunomodulatory effect is assessed by a chemotaxis assay.

4. The method of claim 1, wherein said leporipox virus polypeptide comprises an amino acid sequence which has at least 80% identity to SEQ ID NO: 31.

5. The method of claim 1, wherein said immunomodulation is selected from the group consisting of immunosuppression, immunostimulation, cell proliferation, apoptosis, decreasing T cell stimulation, and decreasing inflammation in a mammal.

6. The method of claim 1, wherein said mammal is a human.

7. The method of claim 1, wherein said mammal is diagnosed with a tumor.

8. The method of claim 7, wherein said tumor is selected from the group consisting of a carcinoma, a plasmacytoma, a lymphoma, and a sarcoma.

9. The method of claim 1, wherein said mammal has a condition selected from the group consisting of acute inflammation, rheumatoid arthritis, transplant rejection, restenosis, asthma, allergies, inflammatory bowel disease, uveitis, psoriasis, atopic dermatitis, bronchial asthma, pollinosis, systemic lupus erythematosus, nephrotic syndrome lupus, multiple sclerosis, myasthenia gravis, type I and type II diabetes mellitus, glomerulonephritis, Hashimoto's thyroiditis, autoimmune hemolytic anemia, autoimmune thrombocytopenic purpura, microbial infection, malignancy and metastasis, autoimmune disease, cirrhosis, endotoxemia, atherosclerosis, reperfusion injury and inflammatory responses, AIDS, cirrhosis of the liver, neurodegeneration, myelodysplastic syndrome, and ischemic injury.

10. A pharmaceutical composition comprising at least one dose of a therapeutically effective amount of a leporipox virus polypeptide or fragment thereof, in a pharmaceutically acceptable carrier, said composition being formulated for the treatment of an immunomodulatory disorder.

11. The pharmaceutical composition of claim 10, wherein said leporipox virus polypeptide comprises an amino acid sequence substantially identical to SEQ ID NO: 31 or fragments and analogs thereof.

12. The pharmaceutical composition of claim 10, wherein said leporipox virus polypeptide comprises an amino acid sequence which has at least 80% identity to SEQ ID NO: 31.

13. A method of detecting a leporipox virus polypeptide in a sample, said method comprising contacting said sample with the antibody of claim 26, and assaying for the binding of said antibody to said polypeptide.

14. The method of claim 13, wherein said leporipox virus polypeptide comprises an amino acid sequence substantially identical to SEQ ID NO: 31 or fragments and analogs thereof.

15. The method of claim 13, wherein said leporipox virus polypeptide comprises an amino acid sequence which has at least 80% identity to SEQ ID NO: 31.
